(19) United States
(12) Patent Application Publication
(10) Pub. No.: US 2017/0145156 A1
(43) Pub. Date: May 25, 2017

(54) Catalysis and Catalysts for Polymerization

(71) Applicants: International Business Machines Corporation, Armonk, NY (US); Board of Trustees of the Leland Stanford Junior University, Stanford, CA (US)

(72) Inventors: Timothy BLAKE, Palo Alto, CA (US); Dylan J. BODAY, Austin, TX (US); Jeannette M. GARCIA, San Leandro, CA (US); James L. HEDRICK, Pleasanton, CA (US); Robert M. WAYMOUTH, Palo Alto, CA (US); Rudy J. WOJTECKI, San Jose, CA (US)

(21) Appl. No.: 14/952,697

(22) Filed: Nov. 25, 2015

(51) Int. Cl. C08G 63/82; C08G 63/08; C07D 487/08; C07C 319/18; C07C 323/25

(52) U.S. Cl. CPC C08G 63/823; C07C 319/18; C07C 323/25; C07D 487/08; C08G 63/08

(57) ABSTRACT

Methods of metal-free synthesis of a compound for catalysis include mixing an aniline of the general structure (1):

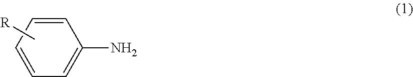

with an aldehyde to form a reaction mixture, and performing a synthesis using the reaction mixture. Each instance of R may be independently an electron withdrawing group. The compound is a thioaminal or a Tröger's base. Polymerization catalysts, or mixtures comprising polymerization catalysts, include the general structure (2):

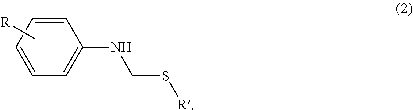

Each instance of R may be independently selected from the group consisting of —H, —F, —CF₃, —NO₂, —Cl, —Br, —I, and nitrile. R′ may be linear or branched alkyl.

### FIELD

Embodiments described herein generally relate to polymerization catalysts and methods of forming the catalysts.

### BACKGROUND

Catalysis is a foundational pillar for sustainable chemical processes, and the discovery of highly active, environmentally benign catalytic processes is a central goal of Green Chemistry. Plastics are ubiquitous and highly useful materials, but their widespread utility and indiscriminate disposal has left an adverse and enduring environmental legacy. Polymers such as polylactides, polycarbonates, and polyesters are biodegradable and biocompatible. Biodegradable and biocompatible polymers offer attractive alternatives to non-biodegradable/non-biocompatible polymers such as polystyrenes. However, syntheses of biodegradable polymers often involve metal-containing catalysts, which have a negative environmental impact.

Therefore, there is a need in the art for biocompatible catalysts and polymer syntheses.

### SUMMARY

The present disclosure describes, a method of synthesizing a compound for catalysis. The method includes mixing an aniline of the general structure (1):

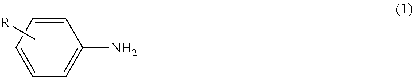

with an aldehyde to form a first reaction mixture. Each instance of R is independently hydrogen or an electron withdrawing group. In some embodiments, at least one instance of R is not hydrogen. The compound is a thioaminal or a Tröger's base.

The present disclosure further describes a compound for polymerization catalysis of the formula:

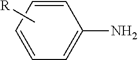

Each instance of R is independently selected from the group consisting of —H, —F, —CF₃, —NO₂, —Cl, —Br, —I, nitrile, and —N(Me)₂.

The present disclosure further describes a compound for polymerization catalysis of the formula:

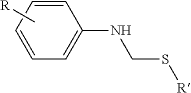

Each instance of R is independently selected from the group consisting of —H, —F, —CF₃, and —NO₂. R′ is linear or branched alkyl.

The present disclosure further describes a compound for polymerization catalysis of the formula:

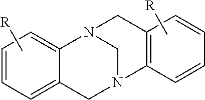

Each instance of R is independently selected from the group consisting of —H, —F, —CF₃, —N(Me)₂, ⁻NO₂.

The present disclosure further describes a method of polymerizing a monomer by mixing a monomer and a catalyst to form a polymer. The monomer may be selected from the group consisting of lactide, carbonate, ester, and mixtures thereof. The polymer has a polydispersity index between about 0.8 to about 1.5 and an Mn or Mw between about 5,000 g/mol to about 25,000 g/mol. The catalyst is selected from the group consisting of:

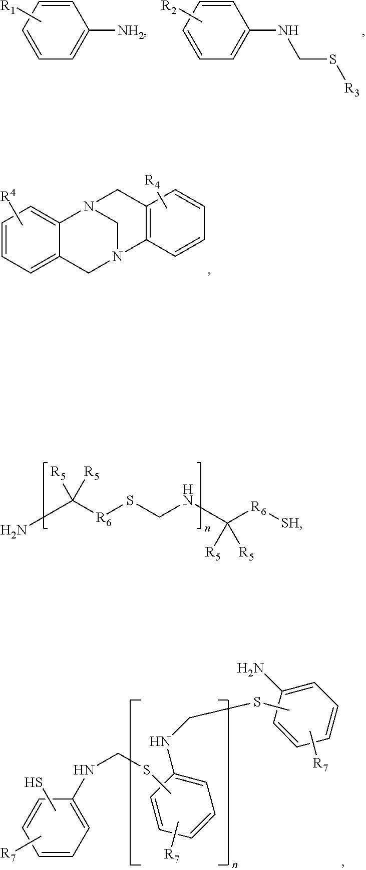

and mixtures thereof. Each instance of R₁ is independently selected from the group consisting of —H, —F, —CF₃, —NO₂, —Cl, —Br, —I, nitrile, and —N(alkyl)₂. Each instance of R₂ is independently selected from the group consisting of H, —F, —CF₃, —NO₂, —Cl, —Br, —I, and nitrile. R₃ is selected from the group consisting of linear or branched alkyl. Each instance of R₄ is independently selected from the group consisting of —H, —F, —CF₃, —N(Me)₂, —NO₂. Each instance of R₅ is independently selected from the group consisting of —H, —F, —CF₃, —NO₂, —Cl, —Br, —I, and nitrile. R₆ is selected from the group consisting of cycloalkyl, alkyl, alkylene glycol, acrylate, and siloxane. Each instance of R₇ is independently selected from the group consisting of —H, —F, —CF₃, —NO₂, —Cl, —Br, —I, nitrile.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the schemes and appended drawings. It is to be noted, however, that the schemes and appended drawings illustrate only typical embodiments of this present disclosure and are therefore not to be considered limiting of its scope, for the present disclosure may admit to other equally effective embodiments.

FIG. 1 illustrates 1-H NMR spectra of p-N,N-dimethylamino-aniline and a Tröger's base (in deuterated trifluoroacetic acid), according to an embodiment.

To facilitate understanding, identical reference numerals have been used, where possible, to designate identical elements that are common to the schemes and figures. The schemes and figures are not drawn to scale and may be simplified for clarity. It is contemplated that elements and features of one embodiment may be beneficially incorporated in other embodiments without further recitation.

### DETAILED DESCRIPTION

Embodiments described herein generally relate to polymerization catalysts and methods of forming the catalysts. Compounds, compositions, and methods described herein may be derived from hexahydrotriazine formation or an intermediate of hexahydrotriazine formation, such as an imine. Mechanistic and theoretical investigations of hexahydrotriazine and thioaminal formation have provided new insights on the diversity of mechanistic pathways for catalyst synthesis. Organocatalytic polymerization reactions and the opportunities of these new insights further facilitate syntheses of macromolecular architectures.

The present disclosure describes methods of synthesizing compounds for catalysis from aniline precursors and formaldehyde or paraformaldehyde. The aniline has the general structure (1):

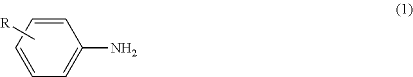

where R may be an electron withdrawing group, and the structure has one, two, three, four, or five R groups. Each instance of R may be independently selected from the group consisting of —H, —F, —CF₃, —NO₂, —Cl, —Br, —I, nitrile, and —N(alkyl)₂. In some embodiments, at least one R is not hydrogen. Alkyl includes methyl, ethyl, propyl, butyl, pentyl, hexyl, heptyl, octyl, nonyl, and decyl. As described herein “Me” means “methyl”.

The present disclosure further describes a compound for polymerization catalysis of the formula:

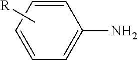

Each instance of R is independently selected from the group consisting of —H, —F, —CF₃, —NO₂, —Cl, —Br, —I, nitrile, and —N(Me)₂.

The products available from the methods described herein include thioaminals and Tröger's base compounds. The products available from the methods described herein include compounds having structures 2-3:

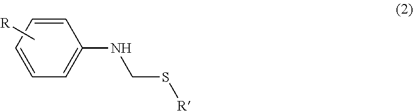

where each instance of R is independently selected from the group consisting of —H, —F, —CF₃, —NO₂, —Cl, —Br, —I, and nitrile. R′ is linear or branched alkyl. In some embodiments, R′ is hexyl.

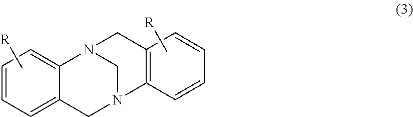

where each instance of R is independently selected from the group consisting of —H, —F, —CF₃, —Cl, —Br, —I, nitrile, and —N(Me)₂.

Compounds having these structures are generally useful as polymerization catalysts. Compounds of structure (2) are generally formed by mixing an aniline of structure (1) with paraformaldehyde and a thiol. Compounds of structure (3) are generally formed by mixing an aniline of structure (1) with paraformaldehyde and an acid.

The equivalents of paraformaldehyde to equivalents of the aniline of general structure (1) may be about 1 in making all the compounds described herein. The first reaction mixture may further include a thiol of the general structure (4):

HS—R′ (4),

wherein R′ is alkyl. The term “alkyl” embraces linear and branched alkyl. Alkyl includes methyl, ethyl, propyl, butyl, pentyl, hexyl, heptyl, octyl, nonyl, decyl, undecyl, dodecyl, tridecyl, tetradecyl, pentadecyl, hexdecyl, heptadecyl, octadecyl, nonadecyl, and eicosyl. Alkyl may be unsubstituted or may be substituted with an electron withdrawing group. Electron withdrawing groups include —F, —CF₃, —NO₂, —Cl, —Br, —I, nitrile, and the like, and combinations thereof.

The first reaction mixture may be heated to between about 80° C. to about 100° C. The first reaction mixture may further include an acid for Tröger's base formation. The first reaction mixture may further include a base for thioaminal formation. The first reaction mixture may further include a solvent selected from the group consisting of N-methyl-pyrrolidone, dimethylsulfoxide, N,N-dimethylformamide, N,N-dimethylacetamide, propylene carbonate, propylene glycol methyl ether acetate, methanol, ethanol, isopropanol, and acetic acid. Methods of the present disclosure further include forming a second reaction mixture comprising a monomer and the compound. The compound may be present in the second reaction mixture between about 0.01 mol % to about 50%. Methods may further include polymerizing the monomer to form a polymer. The monomer may be selected from the group consisting of lactide, carbonate, and ester, and the polymer may be selected from the group consisting of polylactide, polycarbonate, polyester, and mixtures thereof. The polymer may have a polydispersity index of between about 1.00 to about 1.2.

Scheme 1 illustrates catalyst formation under various reaction conditions.

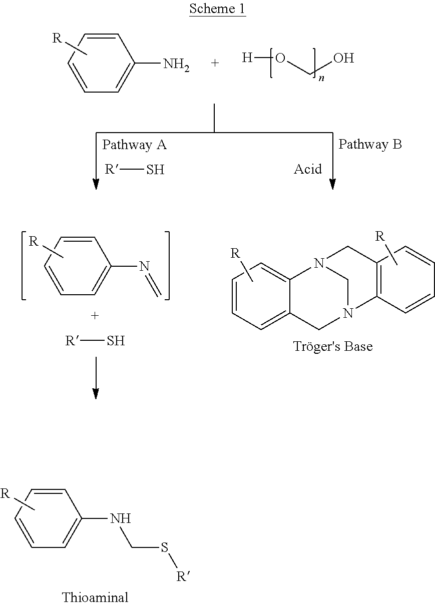

Pathway A shows an aniline compound treated with paraformaldehyde in the presence of a thiol which yields an imine intermediate followed by reaction of the imine with the thiol starting material to yield a thioaminal. The reaction may be performed at room temperature or the reaction may be heated to between about 30° C. to about 120° C., such as between about 80° C. to about 100° C. The reaction of Pathway A may be performed in the presence of a base, such as a weak base. Weak bases include, for example, NaHCO₃ and ammonia.

Pathway B shows an aniline compound treated with paraformaldehyde in the presence of an acid to yield a Tröger's base. Tröger's base formation according to Pathway B may be performed at room temperature or the reaction may be heated to between about 30° C. to about 120° C., such as between about 80° C. to about 100° C.

Equivalents of aldehyde relative to equivalents of an aniline compound may be greater than one for each of Pathway A and Pathway B. In some embodiments, equivalents of aldehyde to equivalents of an aniline compound is about 1. A different aldehyde (i.e., not paraformaldehyde) may be used in addition to or as a replacement of paraformaldehyde. Aldehydes include formaldehyde, acetaldehyde, and polymerized aldehydes such as paraformaldehyde. A ketone, such as acetone, may be used instead of or in addition to an aldehyde. Reactions according to Pathway A and Pathway B may be carried out in a reaction vessel, such as a glass, round-bottom flask, or other suitable vessel. In some embodiments, the vessel is purged with nitrogen or other inert gas prior to a reaction of Pathway A and/or Pathway B. After a reaction has been carried to a stopping point, such as completion of the reaction, vacuum may then be applied to the vessel to remove volatile byproducts and/or solvent. Alternatively, the product may be used in-situ for subsequent polymerization reactions. The starting materials of Pathway A and Pathway B may be obtained from commercial suppliers, such as Sigma-Aldrich, or may be synthesized.

As shown in Scheme 1, an aniline has the general structure (1):

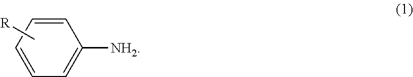

The aniline of general structure (1) may be mono-substituted, di-substituted, tri-substituted, tetra-substituted, or penta-substituted with an R group, each of which may be the same as, or different from, any or all other R groups. One or more R groups of the aniline of general structure (1) may be an electron withdrawing group. Electron withdrawing groups include —F, —CF₃, —NO₂, —Cl, —Br, —I, nitrile, and the like. The one or more R groups of the aniline of general structure (1) form the one or more R groups of the compounds of Scheme 1. One or more electron withdrawing R groups of the compounds promotes catalytic stability, which promotes catalytic activity, such as catalytic turnover, for a polymerization reaction. One or more R groups of the aniline of general structure (1) may be independently selected from the group consisting of —H, —F, —CF₃, —NO₂, —Cl, —Br, —I, nitrile, and —N(Me)₂.

As shown in Scheme 1, a usable thiol has the general structure (4):

HS—R′ (4),

wherein R′ includes at least one carbon. R′ can be an alkyl group, for example, having 1 to 12 carbon atoms (C₁ to C₁₂), such as a hexyl radical. R′ may be linear or branched alkyl. Alkyl may be unsubstituted or may be substituted with an electron withdrawing group. Electron withdrawing groups include —F, —CF₃, —NO₂, —Cl, —Br, —I, nitrile, and the like, and combinations thereof.

Reactions of Pathway A and Pathway B may be carried out in the presence of a solvent, such as an organic solvent. An organic solvent may be polar aprotic. Polar aprotic solvents usable for the methods described herein include N-methyl-pyrrolidone (NMP), dimethylsulfoxide (DMSO), N,N-dimethylformamide (DMF), N,N-dimethylacetamide (DMA), propylene carbonate (PC), propylene glycol methyl ether acetate (PGMEA), and mixtures thereof. An organic solvent may be polar protic. Polar protic solvents may include, for example, methanol, ethanol, isopropanol, acetic acid, and mixtures thereof

In some embodiments, reactions of Pathway A provide quantitative yields, allowing a thioaminal to be used as a catalyst without isolation/purification from any byproducts or starting materials subsequent to thioaminal formation. Reactions of Pathway B may provide quantitative yields, allowing a Tröger's base product to be used as a catalyst without isolation/purification from any byproducts or starting materials subsequent to Tröger's base formation. Alternatively, a Tröger's base product may be purified from the acid starting material by, for example, extraction with water and an organic solvent.

### EXAMPLE 1

### Pathway A Reaction:

Materials: Deuterated solvents (C₆D₆ and CDCl₃) were purchased from Cambridge Isotope Laboratories and dried over activated 3 A molecular sieves then used without further purification. All other substrates were purchased from Aldrich and used without further purification. ¹H-NMR and ¹³C-NMR spectra were recorded on either an Inova 300 MHz, Mercury 400 MHz, or Inova 500 MHz spectrometer. All NMR spectra were taken in CDCl₃ unless otherwise stated. All reactions were performed at 25° C. unless otherwise stated.

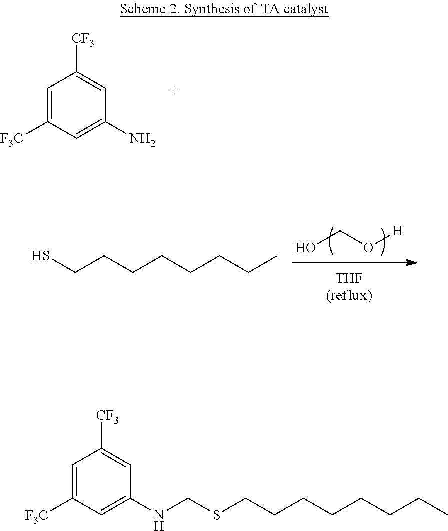

According to the reaction shown in Scheme 2, a flame-dried round-bottom flask was charged with paraformaldehyde (101.2 mg, 3.37 mmol), 3,5-Bis(trifluoromethyl)aniline (380 μL, 2.43 mmol) and octanethiol (435 μL, 2.5 mmol) in tetrahydrofuran (THF) (10 mL). The reaction was heated to reflux and stirred for 16 hours then concentrated in vacuo. The product thioaminal (TA) was used for polymerization experiments with no further purification. ¹H-NMR (300 MHz, (CDCl₃): δ 7.23 (s, 1H), 7.02 (s, 2H), 4.6 (br, 1H, NHCH₂SCH₂), 4.4 (d, 2H, NHCH₂SCH₂, J=6.5 Hz), 2.55 (t, 2H, NHCH₂SCH₂, J=8.3 Hz), 1.6-1.5, (m, 4H*), 1.3-1.2 (m, 13 H*), 0.9 (t, 3.5 H*, J=6.5 Hz); *Some resonances from the in-situ synthesis are partially attributed to impurities: 1.56 ppm (H₂O), 1.25 and 0.85 ppm (grease).

### EXAMPLE 2

### Pathway B Reaction:

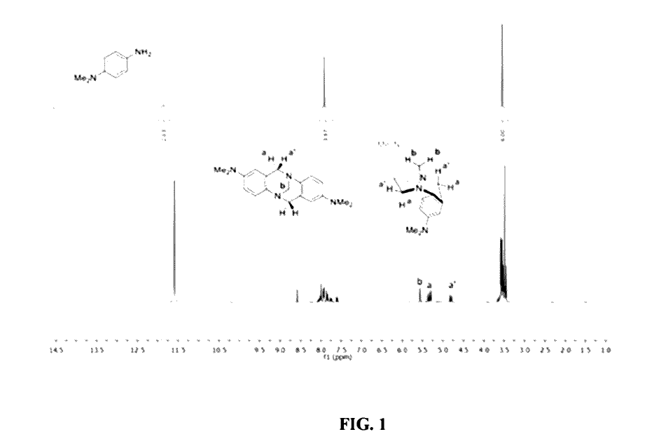

FIG. 1 illustrates 1-H NMR spectra of p-N,N-dimethylamino-aniline and a Tröger's base (in deuterated trifluoroacetic acid: “d-TFA”) of Formula (3) synthesized via Pathway B. Synthesis of the Tröger's base was carried out with p-N,N-dimethylamino-aniline, formaldehyde, and excess trifluoroacetic acid starting materials. As shown in FIG. 1, the Tröger's base gives distinct doublet and singlet methylene proton peaks from about 4.75 ppm to about 5.75 ppm, multiplet aromatic proton peaks from about 7.5 ppm to about 8.0 ppm, and N,N-dimethyl proton peaks from about 3.4 ppm to about 3.75 ppm. These peaks readily distinguish Tröger's base from p-N,N-dimethylamino-aniline starting material and residual formaldehyde starting material.

Syntheses of catalysts by Pathway A or Pathway B represent new synthetic strategies that provide an environmentally attractive, atom-economical, low energy alternative to traditional metal catalyzed synthesis of catalysts.

The present disclosure further describes a method of polymerizing a monomer by mixing a monomer and a catalyst to form a polymer. The monomer may be selected from the group consisting of lactide, carbonate, ester, and mixtures thereof. The polymer has a polydispersity index between about 0.8 to about 1.5 and an Mn or Mw between about 5,000 g/mol to about 25,000 g/mol. The catalyst is selected from the group consisting of:

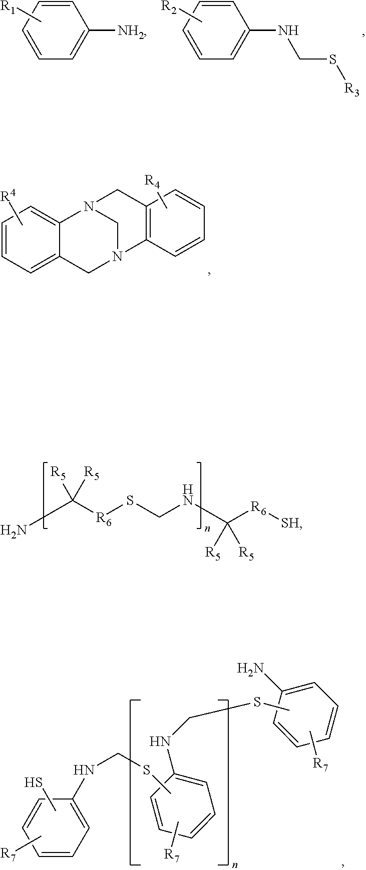

and mixtures thereof. Each instance of R₁ is independently selected from the group consisting of —H, —F, —CF₃, —NO₂, —Cl, —Br, —I, nitrile, and —N(alkyl)₂. Each instance of R₂ is independently selected from the group consisting of H, —F, —CF₃, —NO₂, —Cl, —Br, —I, and nitrile. R₃ is selected from the group consisting of linear or branched alkyl. Each instance of R₄ is independently selected from the group consisting of —H, —F, —CF₃, —N(Me)₂, —NO₂. Each instance of R₅ is independently selected from the group consisting of —H, —F, —CF₃, —NO₂, —Cl, —Br, —I, and nitrile. R₆ is selected from the group consisting of cycloalkyl, alkyl, alkylene glycol, acrylate, and siloxane. Each instance of R₇ is independently selected from the group consisting of —H, —F, —CF₃, —NO₂, —Cl, —Br, —I, nitrile.

Anilines, thioaminals, polythioaminals, and Tröger's base compounds described herein are useful catalysts for various chemical reactions, such as polymerization reactions. Polymerization reactions include, for example, ring opening polymerization reactions, polycondensation reactions, and anionic/zwitterionic polymerizations. Polymerization reactions are preferably ring opening polymerization reactions. Ring opening polymerization reactions include polylactide formation from lactide starting monomers. Other polymerization reactions include polycarbonate formation and polyester formation. In some embodiments, anilines, thioaminals, polythioaminals and Tröger's base compounds may each be present in a polymerization reaction between about 0.01 mol % to about 50 mol %, such as about 0.01 mol % to about 20%, such as about 0.05 mol % to about 10 mol %, such as about 0.05 mol % to about 5 mol %. In this description, “mol %” of catalyst is the molar amount of catalyst divided by the molar amount of polymerization monomer, and this value is multiplied by 100 to obtain a “mol %” value. A co-catalyst may be used in addition to catalysts described herein to form a catalyst system. Co-catalysts include amines such as DBU. In some embodiments, high molecular weight polymers synthesized using catalysts described herein have a polydispersity index (PDI) of between about 0.8 to about 1.5, such as about 1.00 to about 1.2, for example about 1.05. In some embodiments, a “high molecular weight polymer” described herein has a molecular weight (e.g., Mn or Mw) between about 1,000 g/mol to about 50,000 g/mol, such as about 5,000 g/mol to about 25,000 g/mol, such as about 10,000 g/mol to about 20,000 g/mol. In this description, polydispersity index (PDI) is defined as the measure of the width of molecular weight distributions of a polymer. PDI is a value determined by dividing the weight-average molecular weight (Mw) by the number average molecular weight (Mn). In this description, the weight-average molecular weight (Mw) is one measure of molecular weight and is defined as the value obtained by taking all the different-sized polymer molecules in a reaction mixture and calculating their average weight while giving heavier molecules a weight-related bonus (by squaring the molecular mass). Mw is a value determined by the equation:

$${\overline{M}}_{w} = \frac{{\sum }_{i} {N}_{i} {M}_{i}^{2}}{{\sum }_{i} {N}_{i} {M}_{i}} ,$$

where Nᵢ is the number of molecules of molecular mass Mᵢ. The weight-average molecular weight (Mw) of a polymer can be determined by gel permeation chromatography.

In this description, the number average molecular weight (Mn) is another measure of molecular weight and is defined as the value obtained by adding up the weight of each polymer molecule in a reaction mixture and dividing by the number of molecules. Mn is a value determined by the equation:

$${\overline{M}}_{n} = \frac{{\sum }_{i} {N}_{i} {M}_{i}}{{\sum }_{i} {N}_{i}} ,$$

where Nᵢ is the number of molecules of molecular mass Mᵢ. The number average molecular mass (Mn) of a polymer can be determined by gel permeation chromatography.

For thioaminal catalysts synthesized by Pathway A, the thioaminal promotes anionic polymerization at, for example, room temperature in the presence of a deprotonating agent. The thioaminal may also promote substrate activation via the hydrogen bonding capability of, for example, the —NH— moiety of the thioaminal.

### EXAMPLE 4

### Polymerization Reaction with a Catalyst of Structure (1):

Materials. Dialysis bags were purchased from SpectraPor (3500 M_{w}). The solvent (C₇H₈) used for polymerization experiments was dried over activated 3 Å molecular sieves. 1,8-Diazabicycloundec-7-ene (DBU) was purchased from Aldrich and distilled over activated 3Å molecular sieves. δ-Valerolactone (VL) was stirred over CaH₂ then vacuum distilled. Ring-opening polymerization (ROP) experiments were performed in a glove box under nitrogen atmosphere before quenching. ¹H-NMR and ¹³C-NMR spectra were recorded on either an Inova 300 MHz, Mercury 400 MHz, or Inova 500 MHz spectrometer. All NMR spectra were taken in CDCl₃ unless otherwise stated. All reactions were performed at 25° C. unless otherwise stated. Gel permeation chromatography (GPC) was performed in tetrahydrofuran (THF) at a flow rate of 1.0 mL/min on a Waters chromatograph equipped with three Waters columns (300 mm×7.8 mm) connected in series. A Viscotek VE 3580 refractive index detector, Viscotek VE3210 UV/vis detector and Viscotek GPCmax autosampler were employed. The system was calibrated using monodisperse polystyrene standards (Polymer Laboratories).

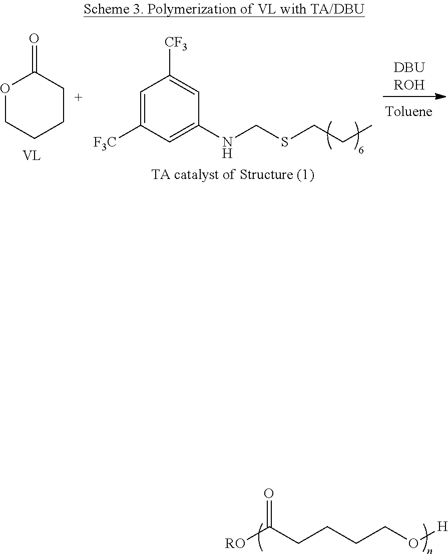

According to the ring-opening polymerization of Scheme 3, a flame-dried vial was charged with VL (53.2 mg, 0.53 mmol) and 0.25 mL toluene. DBU (7.4 mg, 0.05 mmol), TA (10.5 mg, 0.027 mmol) and 1-pyrenebutanol (1.35 mg, 0.005 mmol) in 0.25 mL toluene were added to the reaction mixture. The reaction was allowed to stir for up to 96 hours. Aliquots were taken at 18, 44, 72 and 96 hours, quenching with 1 drop of AcOH. After 96 hours, 100% conversion had been achieved (as determined by NMR spectra), toluene had evaporated and the reaction was dialyzed in dichloromethane against methanol. Removal of solvent under reduced pressure resulted in a clear residue (35.1 mg, 66% yield). GPC (RI): Mn (PDI): 8400 g mol⁻¹ (M_{w}/Mₙ=1.14).

In some embodiments, an aniline may be used a polymerization catalyst. Aniline catalysts include an aniline of general structure (1).

### POLYMERIZATION EXAMPLE

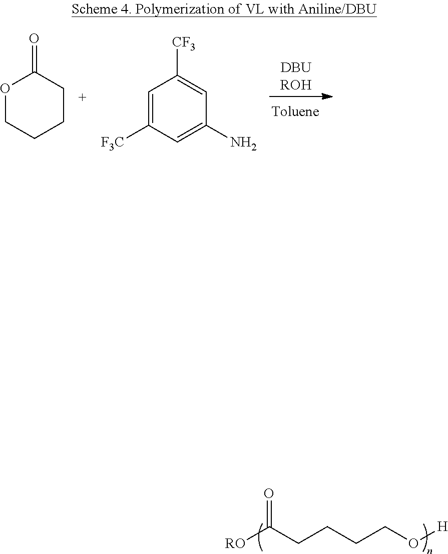

According to the ring-opening polymerization of Scheme 4, a flame-dried vial was charged with VL (51.7 mg, 0.52 mmol), 1-pyrenebutanol (7.0 mg, 0.026 mmol) and DBU (7.4 mg, 0.05 mmol) in 0.4 mL toluene. 3,5-Bis(trifluoromethyl)aniline (4.1 μL in 0.1 mL toluene, 0.026 mmol) was added to the reaction vessel and allowed to stir for up to 96 hours. ¹H-NMR spectra were acquired at 20, 44, 72 and 96 hours, quenching with 1 drop AcOH. At 96 hours the residue was dialyzed in DCM against MeOH resulting in white residue (36.1 mg, 69% yield). GPC (RI): Mn (PDI): 4100 g mol⁻¹ (M_{w}/Mₙ=1.08). Accordingly, the Mn value of the polyester synthesized using the TA catalyst is over twice the Mn value of the polyester synthesized using bis(trifluoromethyl)aniline instead of the TA catalyst.

Non-limiting examples of thioaminals according to structure (2) are shown in Table 1. Each of the chemical compounds of Table 1 has at least one R group and each instance of R throughout Table 1 is independently selected from the group consisting of hydrogen, fluorine, trifluoromethyl, and nitro. R′ is linear or branched alkyl for all the examples of Table 1.

TABLE 1

| Ex. # | Structure |
|---|---|
| 1 | 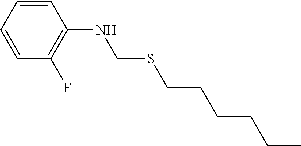 [SMILES: CCCCCCSCNc1ccccc1F] |
| 2 | 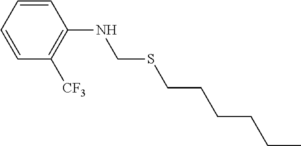 [SMILES: CCCCCCSCNc1ccccc1C(F)(F)F] |
| 3 | 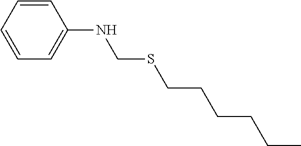 |
| 4 | 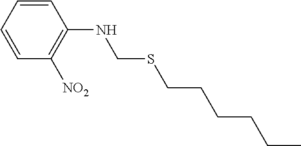 [SMILES: CCCCCCSCNc1ccccc1[N+](=O)[O-]] |
| 5 | 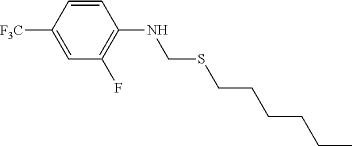 [SMILES: CCCCCCSCNc1ccc(C(F)(F)F)cc1F] |
| 6 | 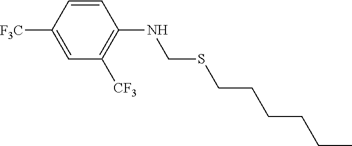 [SMILES: CCCCCCSCNc1ccc(C(F)(F)F)cc1C(F)(F)F] |
| 7 | 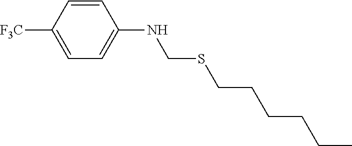 [SMILES: CCCCCCSCNc1ccc(C(F)(F)F)cc1] |
| 8 | 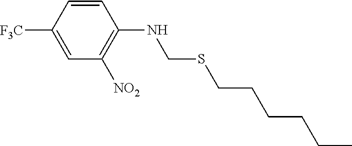 [SMILES: CCCCCCSCNc1ccc(C(F)(F)F)cc1[N+](=O)[O-]] |
| 9 | 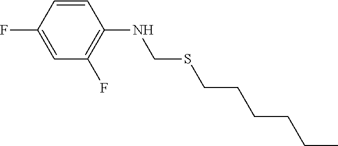 [SMILES: CCCCCCSCNc1ccc(F)cc1F] |
| 10 | 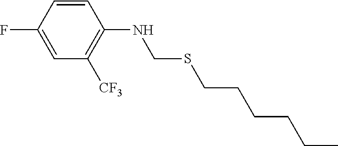 [SMILES: CCCCCCSCNc1ccc(F)cc1C(F)(F)F] |
| 11 | 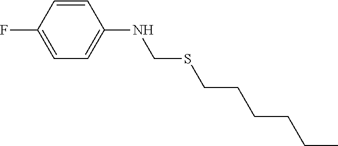 [SMILES: CCCCCCSCNc1ccc(F)cc1] |
| 12 | 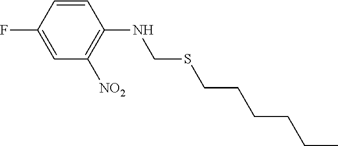 [SMILES: CCCCCCSCNc1ccc(F)cc1[N+](=O)[O-]] |
| 13 | 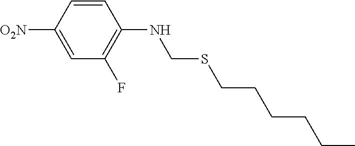 [SMILES: CCCCCCSCNc1ccc([N+](=O)[O-])cc1F] |
| 14 | 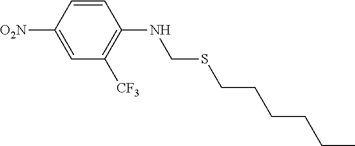 [SMILES: CCCCCCSCNc1ccc([N+](=O)[O-])cc1C(F)(F)F] |
| 15 | 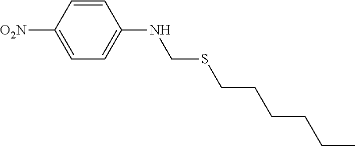 [SMILES: CCCCCCSCNc1ccc([N+](=O)[O-])cc1] |
| 16 | 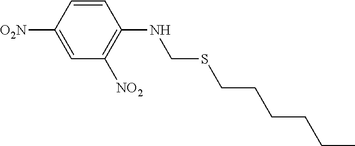 [SMILES: CCCCCCSCNc1ccc([N+](=O)[O-])cc1[N+](=O)[O-]] |
| 17 | 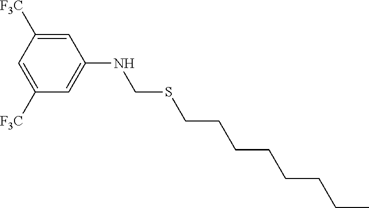 [SMILES: CCCCCCCCSCNc1cc(C(F)(F)F)cc(C(F)(F)F)c1] |
| 18 | 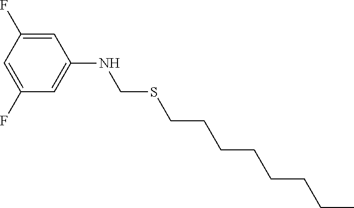 [SMILES: CCCCCCCCSCNc1cc(F)cc(F)c1] |
| 19 | 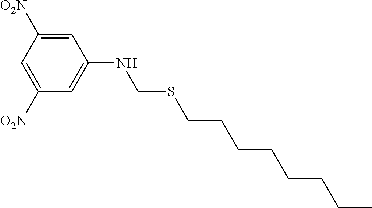 [SMILES: CCCCCCCCSCNc1cc([N+](=O)[O-])cc([N+](=O)[O-])c1] |
| 20 | 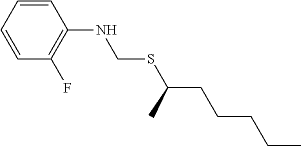 [SMILES: CCCCC[C@@H](C)SCNc1ccccc1F] |
| 21 | 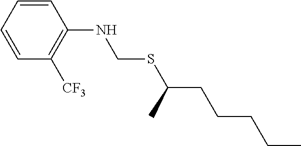 [SMILES: CCCCC[C@@H](C)SCNc1ccccc1C(F)(F)F] |
| 22 | 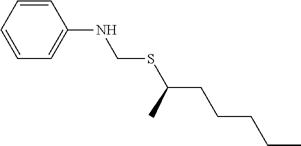 [SMILES: CCCCC[C@@H](C)SCNc1ccccc1] |
| 23 | 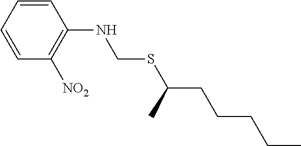 [SMILES: CCCCC[C@@H](C)SCNc1ccccc1[N+](=O)[O-]] |
| 24 | 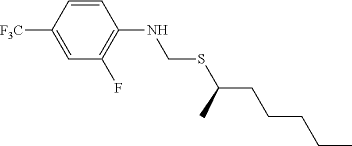 [SMILES: CCCCC[C@@H](C)SCNc1ccc(C(F)(F)F)cc1F] |
| 25 | 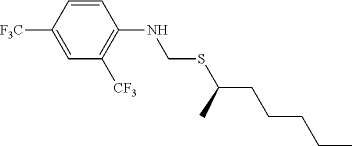 [SMILES: CCCCC[C@@H](C)SCNc1ccc(C(F)(F)F)cc1C(F)(F)F] |
| 26 | 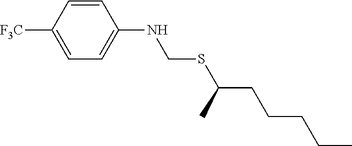 [SMILES: CCCCC[C@@H](C)SCNc1ccc(C(F)(F)F)cc1] |
| 27 | 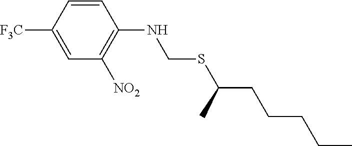 [SMILES: CCCCC[C@@H](C)SCNc1ccc(C(F)(F)F)cc1[N+](=O)[O-]] |
| 28 | 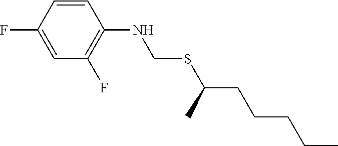 [SMILES: CCCCC[C@@H](C)SCNc1ccc(F)cc1F] |
| 29 | 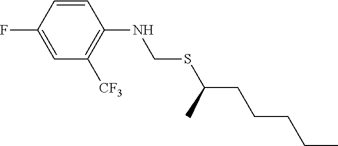 [SMILES: CCCCC[C@@H](C)SCNc1ccc(F)cc1C(F)(F)F] |
| 30 | 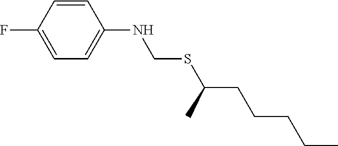 [SMILES: CCCCC[C@@H](C)SCNc1ccc(F)cc1] |
| 31 | 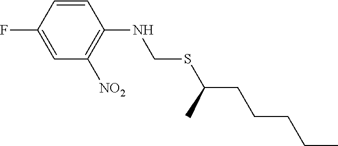 [SMILES: CCCCC[C@@H](C)SCNc1ccc(F)cc1[N+](=O)[O-]] |
| 32 | 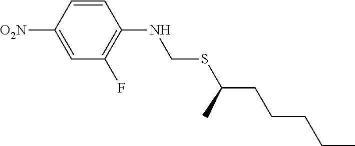 [SMILES: CCCCC[C@@H](C)SCNc1ccc([N+](=O)[O-])cc1F] |
| 33 | 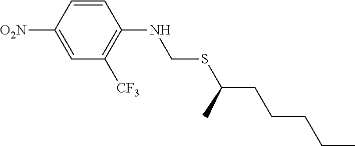 [SMILES: CCCCC[C@@H](C)SCNc1ccc([N+](=O)[O-])cc1C(F)(F)F] |
| 34 | 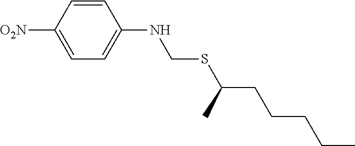 [SMILES: CCCCC[C@@H](C)SCNc1ccc([N+](=O)[O-])cc1] |
| 35 | 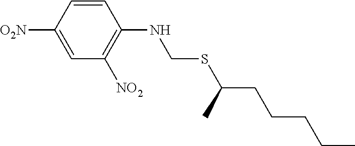 [SMILES: CCCCC[C@@H](C)SCNc1ccc([N+](=O)[O-])cc1[N+](=O)[O-]] |
| 36 | 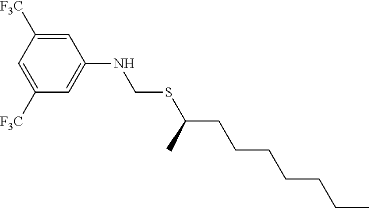 [SMILES: CCCCCCC[C@@H](C)SCNc1cc(C(F)(F)F)cc(C(F)(F)F)c1] |
| 37 | 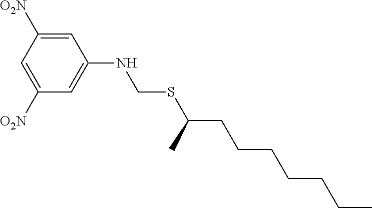 [SMILES: CCCCCCC[C@@H](C)SCNc1cc([N+](=O)[O-])cc([N+](=O)[O-])c1] |
| 38 | 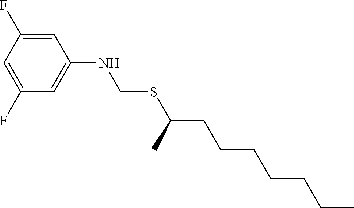 [SMILES: CCCCCCC[C@@H](C)SCNc1cc(F)cc(F)c1] |
| 39 | 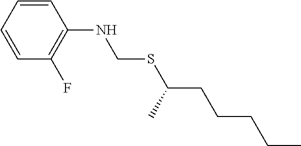 [SMILES: CCCCC[C@H](C)SCNc1ccccc1F] |
| 40 | 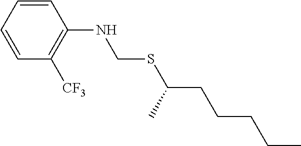 [SMILES: CCCCC[C@H](C)SCNc1ccccc1C(F)(F)F] |
| 41 | 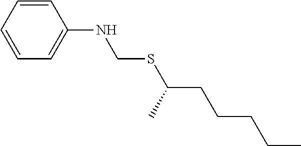 [SMILES: CCCCC[C@H](C)SCNc1ccccc1] |
| 42 | 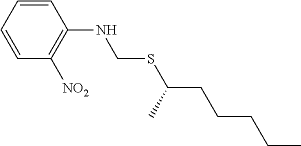 [SMILES: CCCCC[C@H](C)SCNc1ccccc1[N+](=O)[O-]] |
| 43 | 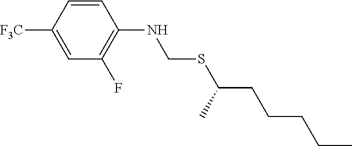 [SMILES: CCCCC[C@H](C)SCNc1ccc(C(F)(F)F)cc1F] |
| 44 | 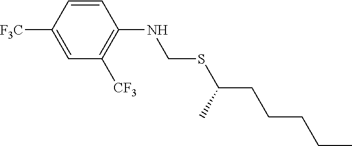 [SMILES: CCCCC[C@H](C)SCNc1ccc(C(F)(F)F)cc1C(F)(F)F] |
| 45 | 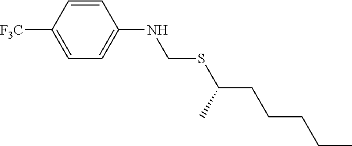 [SMILES: CCCCC[C@H](C)SCNc1ccc(C(F)(F)F)cc1] |
| 46 | 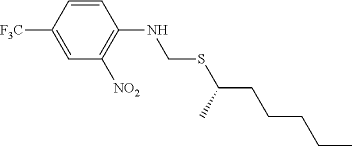 [SMILES: CCCCC[C@H](C)SCNc1ccc(C(F)(F)F)cc1[N+](=O)[O-]] |
| 47 | 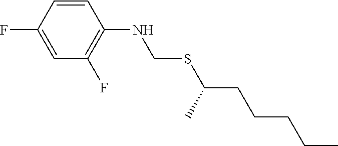 [SMILES: CCCCC[C@H](C)SCNc1ccc(F)cc1F] |
| 48 | 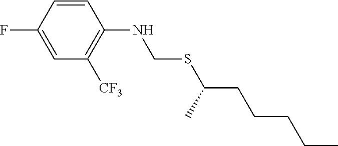 [SMILES: CCCCC[C@H](C)SCNc1ccc(F)cc1C(F)(F)F] |
| 49 | 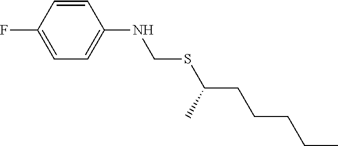 [SMILES: CCCCC[C@H](C)SCNc1ccc(F)cc1] |
| 50 | 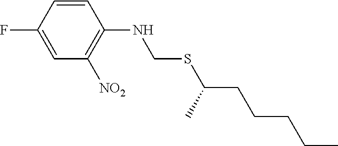 [SMILES: CCCCC[C@H](C)SCNc1ccc(F)cc1[N+](=O)[O-]] |
| 51 | 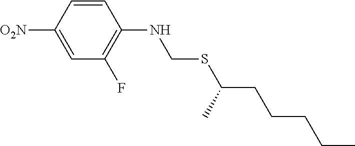 [SMILES: CCCCC[C@H](C)SCNc1ccc([N+](=O)[O-])cc1F] |
| 52 | 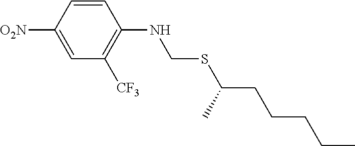 [SMILES: CCCCC[C@H](C)SCNc1ccc([N+](=O)[O-])cc1C(F)(F)F] |
| 53 | 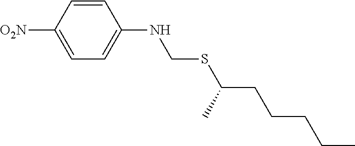 [SMILES: CCCCC[C@H](C)SCNc1ccc([N+](=O)[O-])cc1] |
| 54 | 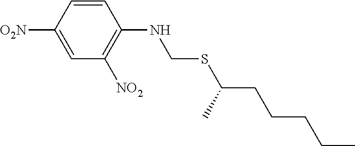 [SMILES: CCCCC[C@H](C)SCNc1ccc([N+](=O)[O-])cc1[N+](=O)[O-]] |
| 55 | 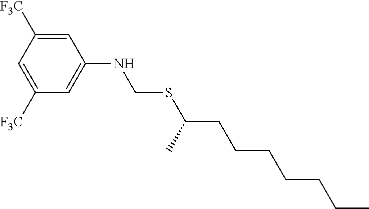 [SMILES: CCCCCCC[C@H](C)SCNc1cc(C(F)(F)F)cc(C(F)(F)F)c1] |
| 56 | 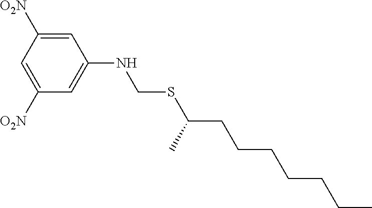 [SMILES: CCCCCCC[C@H](C)SCNc1cc([N+](=O)[O-])cc([N+](=O)[O-])c1] |
| 57 | 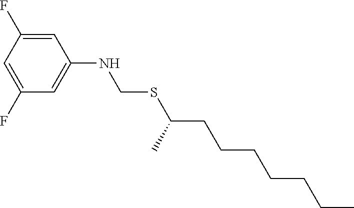 [SMILES: CCCCCCC[C@H](C)SCNc1cc(F)cc(F)c1] |
| 58 | 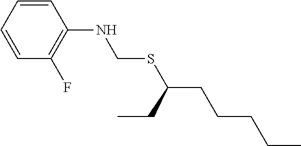 [SMILES: CCCCC[C@@H](CC)SCNc1ccccc1F] |
| 59 | 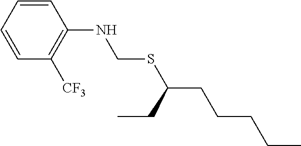 [SMILES: CCCCC[C@@H](CC)SCNc1ccccc1C(F)(F)F] |
| 60 | 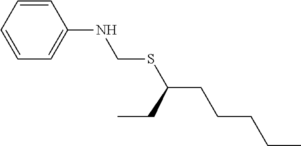 [SMILES: CCCCC[C@@H](CC)SCNc1ccccc1] |
| 61 | 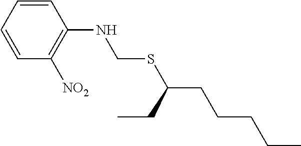 [SMILES: CCCCC[C@@H](CC)SCNc1ccccc1[N+](=O)[O-]] |
| 62 | 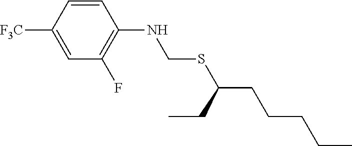 [SMILES: CCCCC[C@@H](CC)SCNc1ccc(C(F)(F)F)cc1F] |
| 63 | 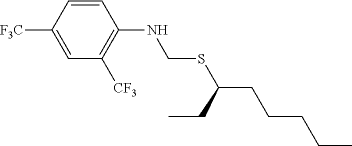 [SMILES: CCCCC[C@@H](CC)SCNc1ccc(C(F)(F)F)cc1C(F)(F)F] |
| 64 | 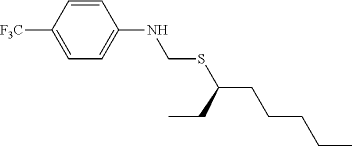 [SMILES: CCCCC[C@@H](CC)SCNc1ccc(C(F)(F)F)cc1] |
| 65 | 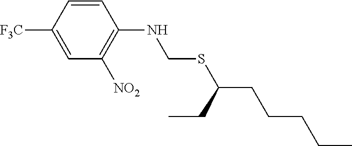 [SMILES: CCCCC[C@@H](CC)SCNc1ccc(C(F)(F)F)cc1[N+](=O)[O-]] |
| 66 | 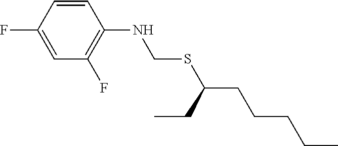 [SMILES: CCCCC[C@@H](CC)SCNc1ccc(F)cc1F] |
| 67 | 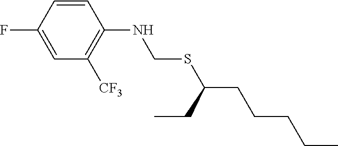 [SMILES: CCCCC[C@@H](CC)SCNc1ccc(F)cc1C(F)(F)F] |
| 68 | 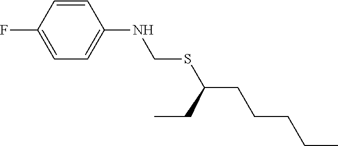 [SMILES: CCCCC[C@@H](CC)SCNc1ccc(F)cc1] |
| 69 | 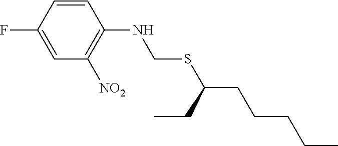 [SMILES: CCCCC[C@@H](CC)SCNc1ccc(F)cc1[N+](=O)[O-]] |
| 70 | 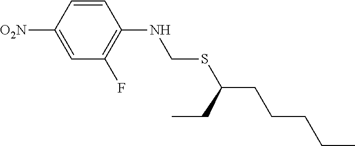 [SMILES: CCCCC[C@@H](CC)SCNc1ccc([N+](=O)[O-])cc1F] |
| 71 | 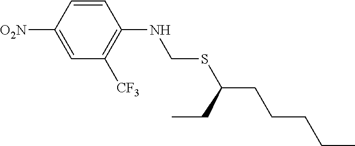 [SMILES: CCCCC[C@@H](CC)SCNc1ccc([N+](=O)[O-])cc1C(F)(F)F] |
| 72 | 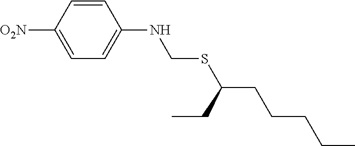 [SMILES: CCCCC[C@@H](CC)SCNc1ccc([N+](=O)[O-])cc1] |
| 73 | 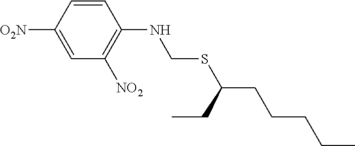 [SMILES: CCCCC[C@@H](CC)SCNc1ccc([N+](=O)[O-])cc1[N+](=O)[O-]] |
| 74 | 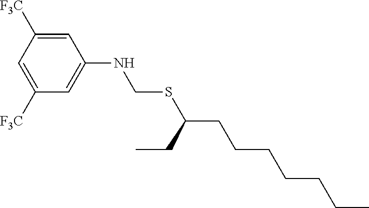 [SMILES: CCCCCCC[C@@H](CC)SCNc1cc(C(F)(F)F)cc(C(F)(F)F)c1] |
| 75 | 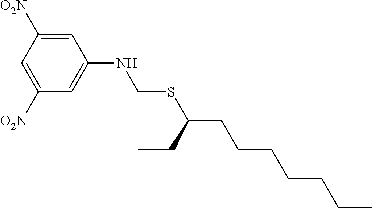 [SMILES: CCCCCCC[C@@H](CC)SCNc1cc([N+](=O)[O-])cc([N+](=O)[O-])c1] |
| 76 | 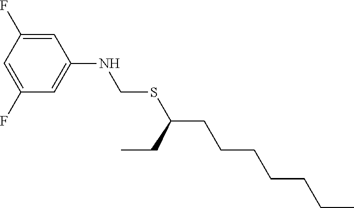 [SMILES: CCCCCCC[C@@H](CC)SCNc1cc(F)cc(F)c1] |
| 77 | 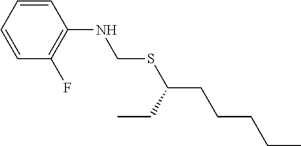 [SMILES: CCCCC[C@H](CC)SCNc1ccccc1F] |
| 78 | 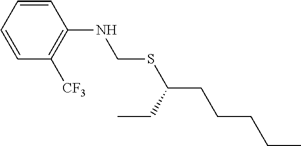 [SMILES: CCCCC[C@H](CC)SCNc1ccccc1C(F)(F)F] |
| 79 | 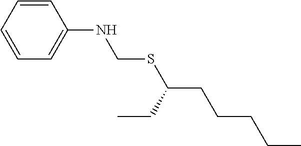 [SMILES: CCCCC[C@H](CC)SCNc1ccccc1] |
| 80 | 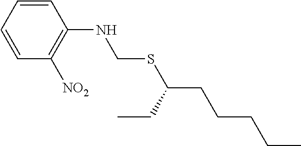 [SMILES: CCCCC[C@H](CC)SCNc1ccccc1[N+](=O)[O-]] |
| 81 | 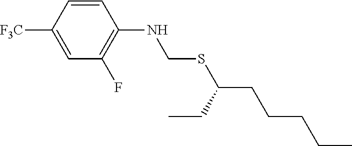 [SMILES: CCCCC[C@H](CC)SCNc1ccc(C(F)(F)F)cc1F] |
| 82 | 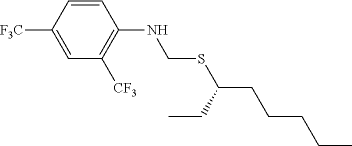 [SMILES: CCCCC[C@H](CC)SCNc1ccc(C(F)(F)F)cc1C(F)(F)F] |
| 83 | 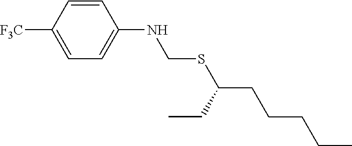 [SMILES: CCCCC[C@H](CC)SCNc1ccc(C(F)(F)F)cc1] |
| 84 | 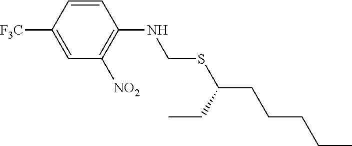 [SMILES: CCCCC[C@H](CC)SCNc1ccc(C(F)(F)F)cc1[N+](=O)[O-]] |
| 85 | 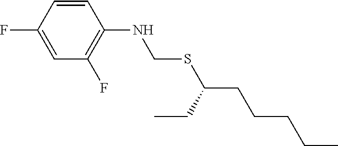 [SMILES: CCCCC[C@H](CC)SCNc1ccc(F)cc1F] |
| 86 | 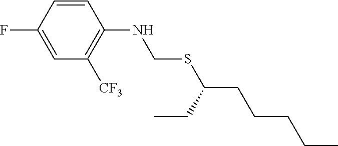 [SMILES: CCCCC[C@H](CC)SCNc1ccc(F)cc1C(F)(F)F] |
| 87 | 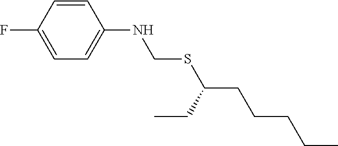 [SMILES: CCCCC[C@H](CC)SCNc1ccc(F)cc1] |
| 88 | 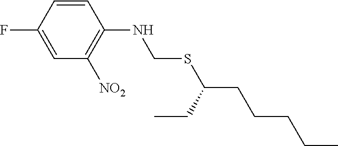 [SMILES: CCCCC[C@H](CC)SCNc1ccc(F)cc1[N+](=O)[O-]] |
| 89 | 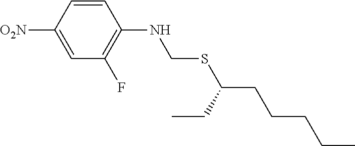 [SMILES: CCCCC[C@H](CC)SCNc1ccc([N+](=O)[O-])cc1F] |
| 90 | 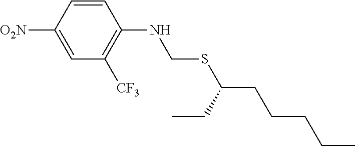 [SMILES: CCCCC[C@H](CC)SCNc1ccc([N+](=O)[O-])cc1C(F)(F)F] |
| 91 | 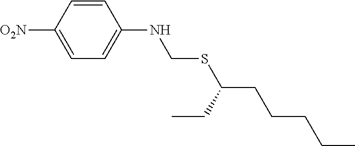 [SMILES: CCCCC[C@H](CC)SCNc1ccc([N+](=O)[O-])cc1] |
| 92 | 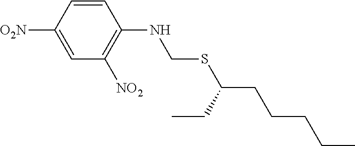 [SMILES: CCCCC[C@H](CC)SCNc1ccc([N+](=O)[O-])cc1[N+](=O)[O-]] |
| 93 | 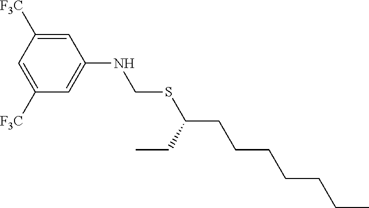 [SMILES: CCCCCCC[C@H](CC)SCNc1cc(C(F)(F)F)cc(C(F)(F)F)c1] |
| 94 | 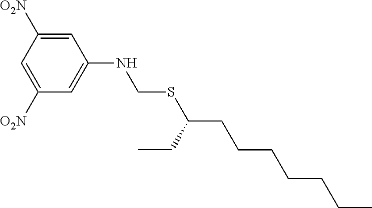 [SMILES: CCCCCCC[C@H](CC)SCNc1cc([N+](=O)[O-])cc([N+](=O)[O-])c1] |
| 95 | 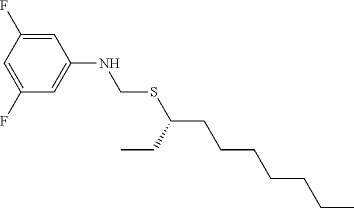 [SMILES: CCCCCCC[C@H](CC)SCNc1cc(F)cc(F)c1] |

Non-limiting examples of Tröger's base according to structure (3) are shown in Table 2. Each of the chemical compounds of Table 2 has at least one R group and each instance of R throughout Table 2 is independently selected from the group consisting of hydrogen, fluorine, trifluoromethyl, nitro, and dimethylamino.

TABLE 2

| Ex. # | Structure |
|---|---|
| 1 | 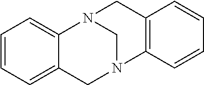 [SMILES: c1ccc2c(c1)CN1CN2Cc2ccccc21] |
| 2 | 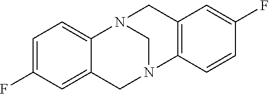 [SMILES: Fc1ccc2c(c1)CN1CN2Cc2cc(F)ccc21] |
| 3 | 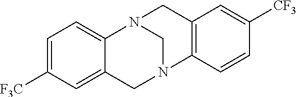 [SMILES: FC(F)(F)c1ccc2c(c1)CN1CN2Cc2cc(C(F)(F)F)ccc21] |
| 4 | 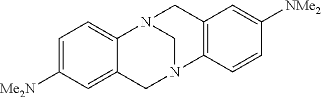 [SMILES: CN(C)c1ccc2c(c1)CN1CN2Cc2cc(N(C)C)ccc21] |
| 5 | 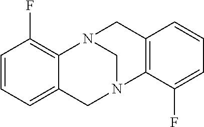 [SMILES: Fc1cccc2c1N1Cc3cccc(F)c3N(C2)C1] |
| 6 | 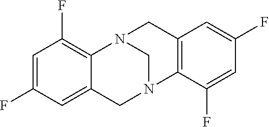 [SMILES: Fc1cc(F)c2c(c1)CN1CN2Cc2cc(F)cc(F)c21] |
| 7 | 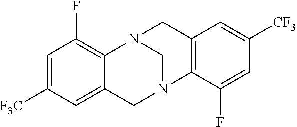 [SMILES: Fc1cc(C(F)(F)F)cc2c1N1Cc3cc(C(F)(F)F)cc(F)c3N(C2)C1] |
| 8 | 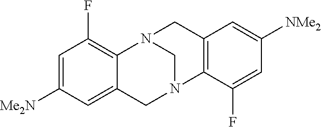 [SMILES: CN(C)c1cc(F)c2c(c1)CN1CN2Cc2cc(N(C)C)cc(F)c21] |
| 9 | 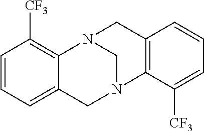 [SMILES: FC(F)(F)c1cccc2c1N1Cc3cccc(C(F)(F)F)c3N(C2)C1] |
| 10 | 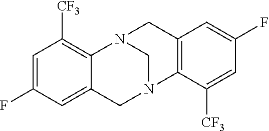 [SMILES: Fc1cc2c(c(C(F)(F)F)c1)N1Cc3cc(F)cc(C(F)(F)F)c3N(C2)C1] |
| 11 | 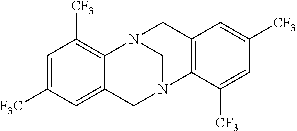 [SMILES: FC(F)(F)c1cc2c(c(C(F)(F)F)c1)N1Cc3cc(C(F)(F)F)cc(C(F)(F)F)c3N(C2)C1] |
| 12 | 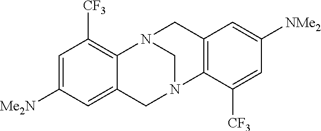 [SMILES: CN(C)c1cc2c(c(C(F)(F)F)c1)N1Cc3cc(N(C)C)cc(C(F)(F)F)c3N(C2)C1] |
| 13 | 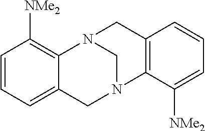 [SMILES: CN(C)c1cccc2c1N1Cc3cccc(N(C)C)c3N(C2)C1] |
| 14 | 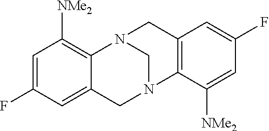 [SMILES: CN(C)c1cc(F)cc2c1N1Cc3cc(F)cc(N(C)C)c3N(C2)C1] |
| 15 | 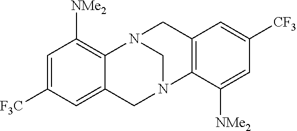 [SMILES: CN(C)c1cc(C(F)(F)F)cc2c1N1Cc3cc(C(F)(F)F)cc(N(C)C)c3N(C2)C1] |
| 16 | 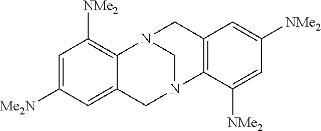 [SMILES: CN(C)c1cc2c(c(N(C)C)c1)N1Cc3cc(N(C)C)cc(N(C)C)c3N(C2)C1] |
| 17 | 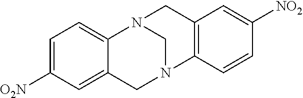 [SMILES: O=[N+]([O-])c1ccc2c(c1)CN1CN2Cc2cc([N+](=O)[O-])ccc21] |
| 18 | 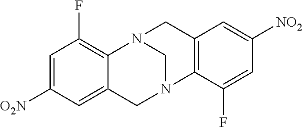 [SMILES: O=[N+]([O-])c1cc(F)c2c(c1)CN1CN2Cc2cc([N+](=O)[O-])cc(F)c21] |
| 19 | 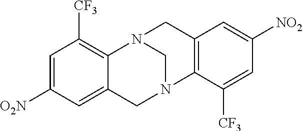 [SMILES: O=[N+]([O-])c1cc2c(c(C(F)(F)F)c1)N1Cc3cc([N+](=O)[O-])cc(C(F)(F)F)c3N(C2)C1] |
| 20 | 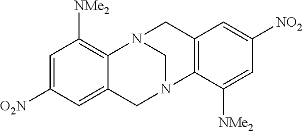 [SMILES: CN(C)c1cc([N+](=O)[O-])cc2c1N1Cc3cc([N+](=O)[O-])cc(N(C)C)c3N(C2)C1] |
| 21 | 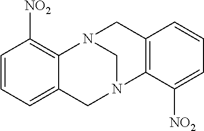 [SMILES: O=[N+]([O-])c1cccc2c1N1Cc3cccc([N+](=O)[O-])c3N(C2)C1] |
| 22 | 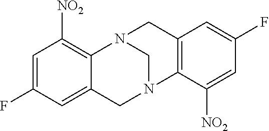 [SMILES: O=[N+]([O-])c1cc(F)cc2c1N1Cc3cc(F)cc([N+](=O)[O-])c3N(C2)C1] |
| 23 | 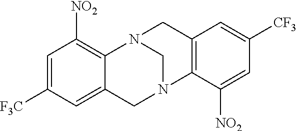 [SMILES: O=[N+]([O-])c1cc(C(F)(F)F)cc2c1N1Cc3cc(C(F)(F)F)cc([N+](=O)[O-])c3N(C2)C1] |
| 24 | 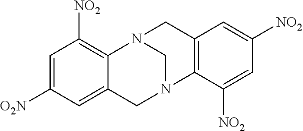 [SMILES: O=[N+]([O-])c1cc2c(c([N+](=O)[O-])c1)N1Cc3cc([N+](=O)[O-])cc([N+](=O)[O-])c3N(C2)C1] |
| 25 | 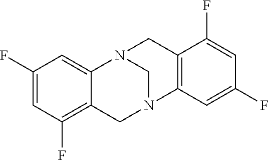 [SMILES: Fc1cc(F)c2c(c1)N1Cc3c(F)cc(F)cc3N(C2)C1] |
| 26 | 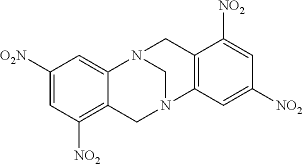 [SMILES: O=[N+]([O-])c1cc2c(c([N+](=O)[O-])c1)CN1CN2Cc2c1cc([N+](=O)[O-])cc2[N+](=O)[O-]] |
| 27 | 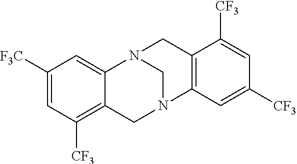 [SMILES: FC(F)(F)c1cc2c(c(C(F)(F)F)c1)CN1CN2Cc2c1cc(C(F)(F)F)cc2C(F)(F)F] |
| 28 | 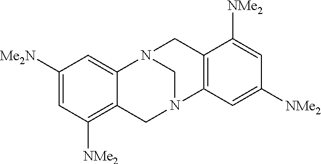 [SMILES: CN(C)c1cc(N(C)C)c2c(c1)N1Cc3c(N(C)C)cc(N(C)C)cc3N(C2)C1] |

In some embodiments, catalysts include aliphatic polythioaminals of general structure (5):

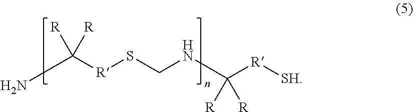

In some embodiments, catalysts include aromatic polythioaminals of the general structure:

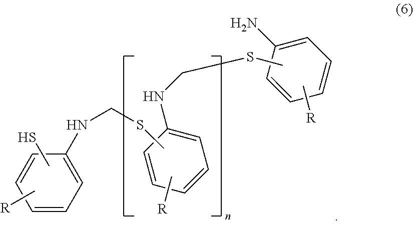

In some embodiments, the —NH— moiety of general structure (6) is para- to the —S— moiety. In some embodiments, at least one of the electron withdrawing groups is ortho- and/or para- to the —NH— moiety.

Aliphatic and aromatic polythioaminals may be synthesized by mixing amino thiol monomers with paraformaldehyde. In some embodiments, the reaction is performed in a solvent. The aliphatic and aromatic amino thiol starting material (and accordingly each instance of R of the polythioaminal product) may be substituted where each instance of R is independently selected from the group consisting of —H, —F, —CF₃, —NO₂, —Cl, —Br, —I, nitrile and the like, and mixtures thereof. For aliphatic polythioaminals, R′ is a linker moiety. Each instance of R′ is independently selected from the group consisting of cycloalkyl, alkyl, alkylene glycol, acrylate, and siloxane. In some embodiments, alkylene glycol is polyalkylene glycol, acrylate is polyacrylate, and siloxane is polysiloxane. In some embodiments, each instance of R′ is independently selected from the group consisting of cyclohexyl, n-butyl, polyethylene glycol 400, polymethylacrylate, and polydimethylsiloxane. Substrates include a polymer bead, silica particle or surface. As described herein, polyethylene glycol 400 is polyethylene glycol with a number average molecular weight (Mn) value of 400 g/mol. Accordingly, a polyethylene glycol moiety may be shown as follows:

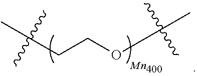

An aliphatic polythioaminal or aromatic polythioaminal may be linked to a substrate by mixing an amino-substituted substrate with an aliphatic amino thiol and paraformaldehyde. A substrate may be flat or round. A substrate may include an outer surface functionalized with one or more R moieties, where each instance of R is selected from the group consisting of —H, —F, —CF₃,—NO₂, —Cl, —Br, —I, nitrile, and the like, and mixtures thereof. Additionally or alternatively, a substrate may be thiol-substituted with one or more thiol moieties. In embodiments where a substrate is thiol-substituted, a thiol-substituted substrate may react with an imine by nucleophilic addition of the thiol nucleophile of the substrate with an electrophilic carbon of an imine (of an aliphatic thiol imine monomer or imine-containing terminus of an aliphatic polythioaminal).

In some embodiments, ‘n’ of the polythioaminal moiety of a polythioaminal-substituted substrate is an integer such that the number average molecular weight (Mn) or weight average molecular weight (Mw) of the polythioaminal moiety is between about 5,500 to about 40,000, such as between about 10,000 to about 25,000, between about 15,000 to about 20,000.

Aliphatic polythioaminals and aromatic polythioaminals described herein are useful as polymerization catalysts, as shown in Scheme 5:

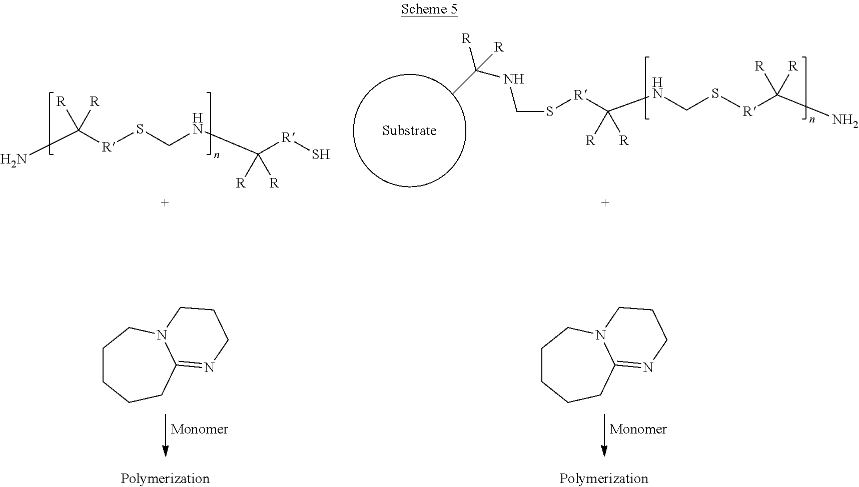

The reactions shown in Scheme 5 may be carried out at room temperature or the reaction may be heated to between about 30° C. to about 120° C., such as between about 50° C. to about 110° C., such as between about 85° C. to about 100° C. The reaction may be performed in the presence of a base, such as a weak base. Weak bases include, for example, NaHCO₃ and ammonia.

As shown in Scheme 5, a polythioaminal promotes anionic polymerization at, for example, room temperature in the presence of a deprotonating agent, such as 1,8-Diazabicycloundec-7-ene (DBU). In some embodiments, DBU is mixed with a polythiaminal catalyst to promote anion formation, followed by addition of a monomer for polymer synthesis. Polythioaminals described herein may be used as catalysts for polymerization reactions, such as ring opening polymerization of lactides to form polylactides. Other polymerization reactions (catalyzed by polythioaminals described herein) include polymethacrylate, poly-trimethylene carbonate, and polyester. In some embodiments, polymers synthesized using polythioaminal catalysts described herein have a polydispersity index (PDI) of between about 1.00 to about 1.2, for example about 1.05.

Compounds of general structures (1)-(6) of the present disclosure can exist in tautomeric, geometric or stereoisomeric forms. Ester, metabolite, oxime, onium, hydrate, solvate and N-oxide forms of compounds of general structures (1)-(6) are also embraced by the present disclosure. The present disclosure considers all such compounds, including, but not limited to, cis- and trans-geometric isomers (Z- and E-geometric isomers), R- and S-enantiomers, diastereomers, d-isomers, 1-isomers, atropisomers, epimers, conformers, rotamers, mixtures of isomers and racemates thereof, as falling within the scope of the present disclosure. Some of the compounds described herein contain one or more stereocenters and are meant to include R, S and mixtures of R and S forms for each stereocenter present.

Compounds and syntheses described herein allow for both biocompatible catalysts and biocompatible polymer syntheses. Syntheses described herein are “metal-free”. Furthermore, syntheses described herein provide catalysts that contain electron withdrawing groups. Catalysts described herein, as well as the electron withdrawing moieties of the catalysts, promote catalytic stability. The catalytic stability promotes catalytic activity, such as catalytic turnover, for a polymerization reaction. In some embodiments, catalyst syntheses described herein provide quantitative yields, allowing a reaction product to be used as a catalyst without isolation/purification from any byproducts or starting materials subsequent to reaction product formation. Catalysts described herein may be used as catalysts for polymerization reactions, such as ring opening polymerization of lactides to form polylactides. In some embodiments, polymers synthesized using catalysts described herein have a polydispersity index (PDI) of between about 1.00 to about 1.2, for example about 1.05.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

While the foregoing is directed to embodiments of the present invention, other and further embodiments of the invention may be devised without departing from the basic scope thereof, and the scope thereof is determined by the claims that follow.

1. A method of synthesizing a compound for catalysis, comprising:
mixing an aniline of the general structure (1): 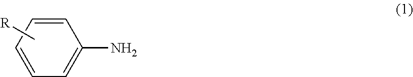
with an aldehyde to form a first reaction mixture, wherein each instance of R is an electron withdrawing group, and performing a synthesis using the reaction mixture to form a compound that is a thioaminal or a Tröger's base.

2. The method of claim 1, wherein the synthesis comprises:
forming a second reaction mixture comprising the compound and one or more monomers selected from the group consisting of lactide, carbonate, and ester, wherein the compound is present between about 0.01 mol % to about 50 mol %; and
polymerizing the one or more monomers to form a polymer selected from the group consisting of polylactide, polycarbonate, polyester, and mixtures thereof.

3. The method of claim 1, wherein the aldehyde is paraformaldehyde.

4. The method of claim 1, further comprising adding to the reaction mixture a thiol of the general structure (2):
HS—R′ (2),
wherein R′ is linear or branched alkyl.

5. The method of claim 1, wherein the synthesis comprises heating the first reaction mixture to between about 80° C. to about 100° C.

6. The method of claim 1, further comprising adding a base to the first reaction mixture.

7. The method of claim 1, further comprising adding an acid to the first reaction mixture.

8. The method of claim 3, wherein a ratio of equivalents of paraformaldehyde to equivalents of the aniline in the first reaction mixture is about 1.

9. The method of claim 1, wherein the electron withdrawing group is selected from the group consisting of —H, —F, —CF₃, —NO₂ and —N(Me)₂.

10. The method of claim 1, further comprising adding a solvent selected from the group consisting of N-methyl-pyrrolidone, dimethylsulfoxide, N,N-dimethylformamide, N,N-dimethylacetamide, propylene carbonate, propylene glycol methyl ether acetate, methanol, ethanol, isopropanol, and acetic acid to the first reaction mixture.

11. The method of claim 2, wherein the polymer has a polydispersity index of between about 1.00 to about 1.2.

12. The method of claim 1, wherein the compound is selected from the group consisting of:

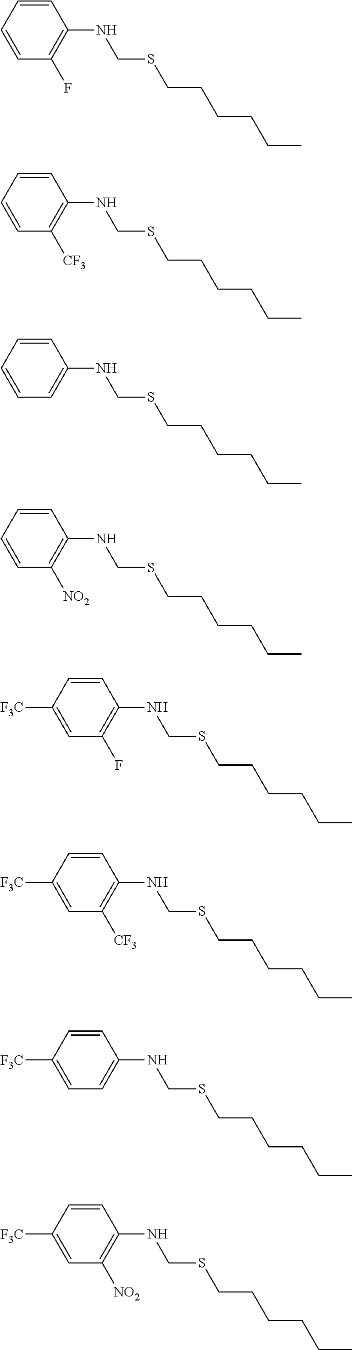

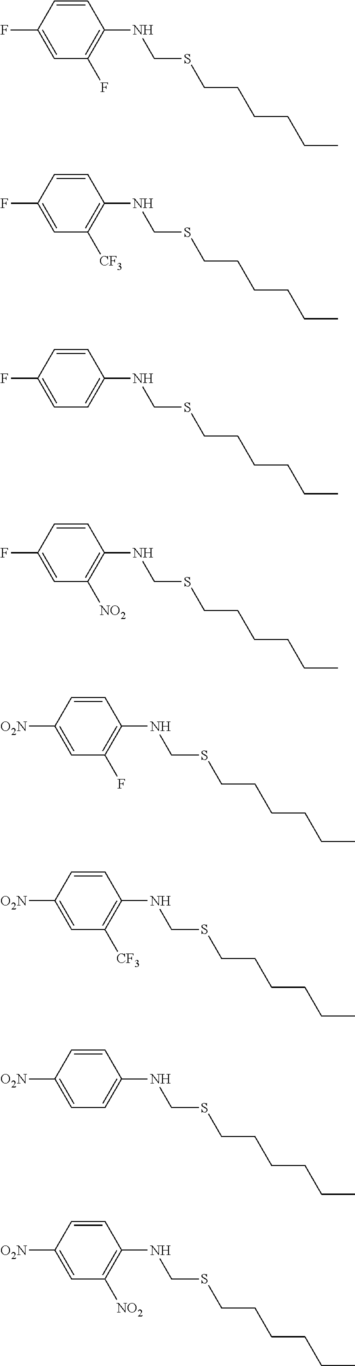

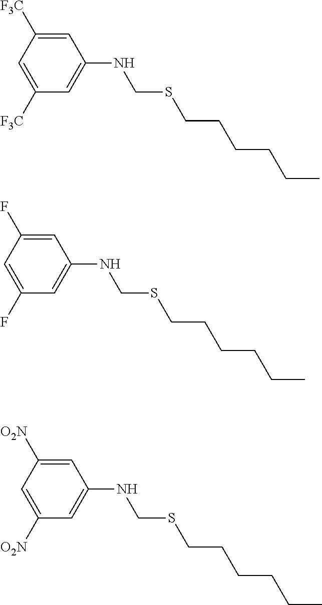 [SMILES: CCCCCCSCNc1cc(C(F)(F)F)cc(C(F)(F)F)c1 ; CCCCCCSCNc1cc(F)cc(F)c1 ; CCCCCCSCNc1cc([N+](=O)[O-])cc([N+](=O)[O-])c1]

13. The method of claim 1, wherein the compound is selected from the group consisting of:

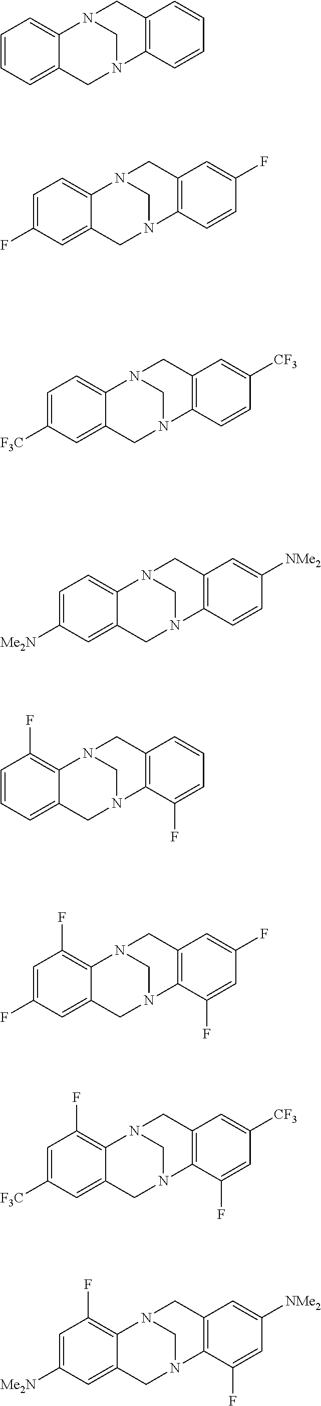

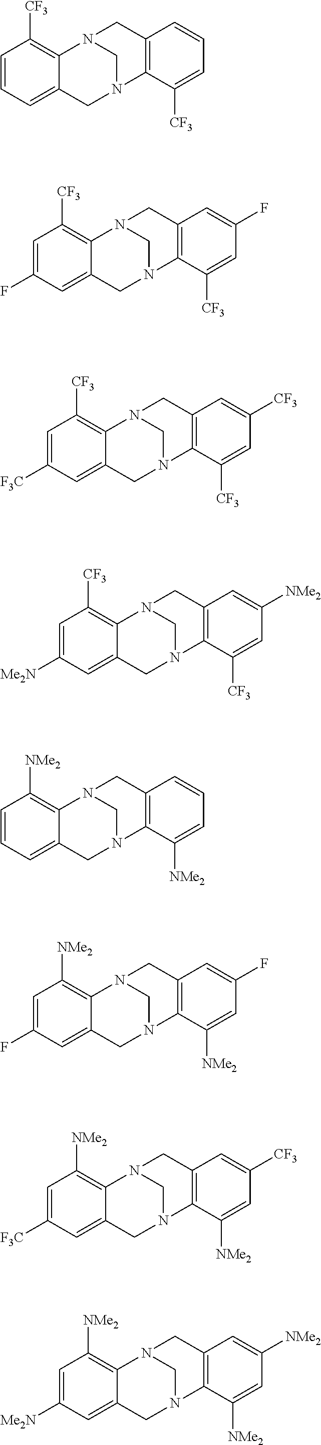

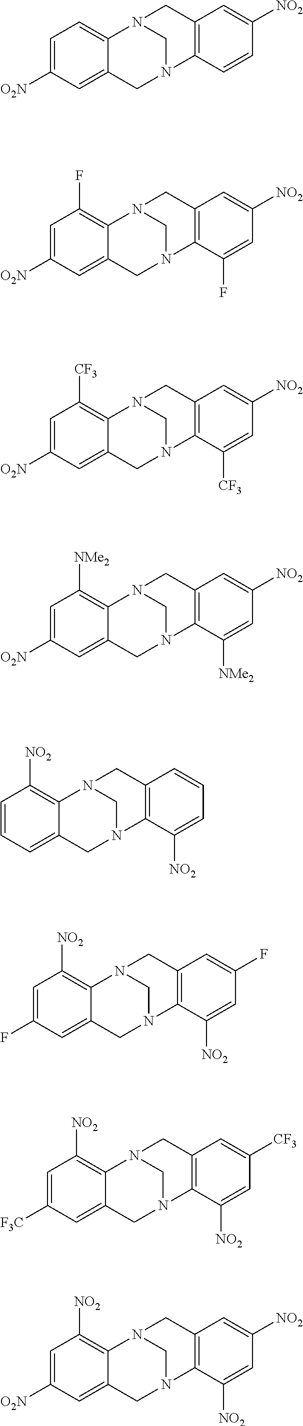

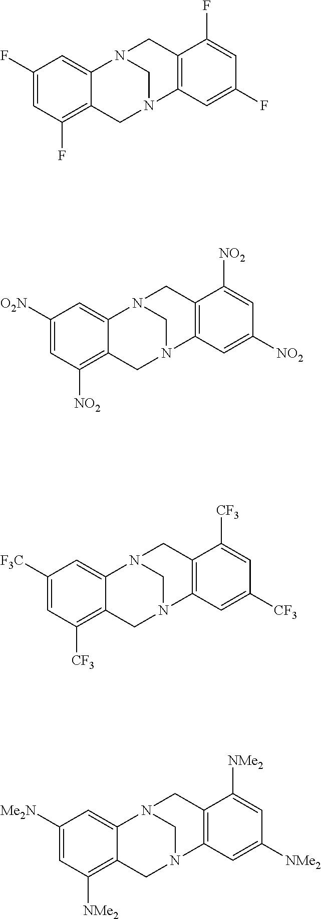 [SMILES: Fc1cc(F)c2c(c1)N1Cc3c(F)cc(F)cc3N(C2)C1 ; O=[N+]([O-])c1cc2c(c([N+](=O)[O-])c1)CN1CN2Cc2c1cc([N+](=O)[O-])cc2[N+](=O)[O-] ; FC(F)(F)c1cc2c(c(C(F)(F)F)c1)CN1CN2Cc2c1cc(C(F)(F)F)cc2C(F)(F)F ; CN(C)c1cc(N(C)C)c2c(c1)N1Cc3c(N(C)C)cc(N(C)C)cc3N(C2)C1]

14. A compound for polymerization catalysis, of the formula: 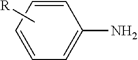
wherein each instance of R is independently selected from the group consisting of —H, —F, —CF₃, —NO₂, —Cl, —Br, —I, nitrile, and —N(Me)₂.

15. A catalyst system comprising 1,8-Diazabicycloundec-7-ene and the compound of claim 14.

16. An adduct of the compound of claim 14 of the structure: 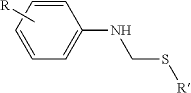
wherein each instance of R is independently selected from the group consisting of —H, —F, —CF₃, and —NO₂, and wherein R′ is linear or branched alkyl.

17. The compound of claim 16, wherein R′ is hexyl.

18. A catalyst system comprising 1,8-Diazabicycloundec-7-ene and the compound of claim 16.

19. A reaction mixture comprising the compound of claim 16 and one or more monomers selected from the group consisting of lactide, carbonate, and ester.

20. The compound of claim 16, which is selected from the group consisting of:

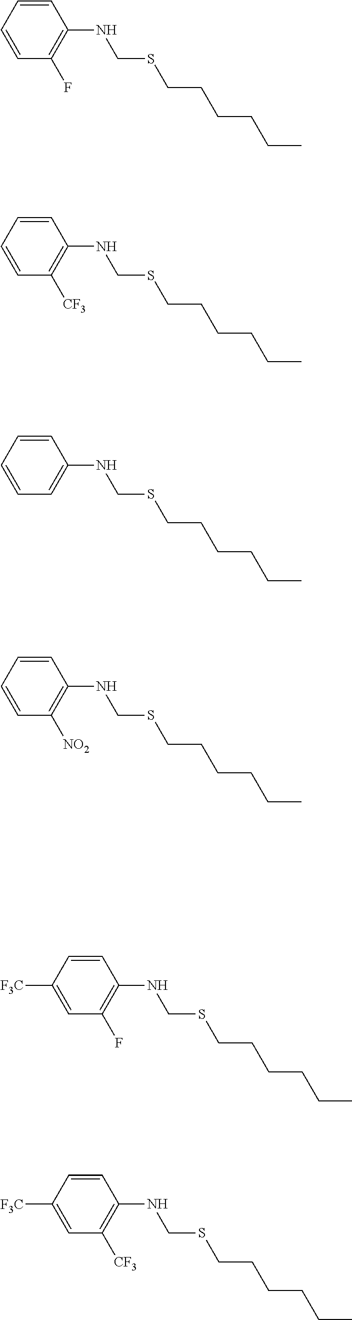 [SMILES: CCCCCCSCNc1ccccc1F ; CCCCCCSCNc1ccccc1C(F)(F)F ; CCCCCCSCNc1ccccc1 ; CCCCCCSCNc1ccccc1[N+](=O)[O-] ; CCCCCCSCNc1ccc(C(F)(F)F)cc1F ; CCCCCCSCNc1ccc(C(F)(F)F)cc1C(F)(F)F]

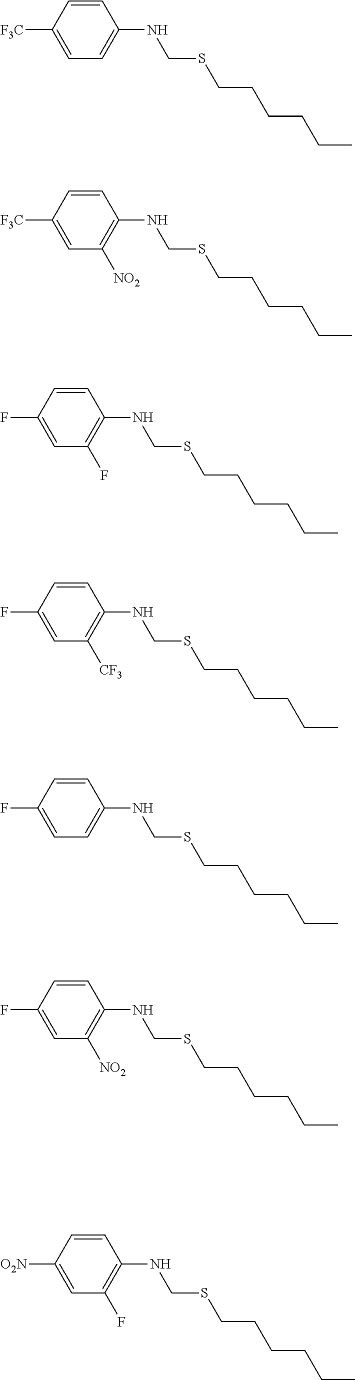

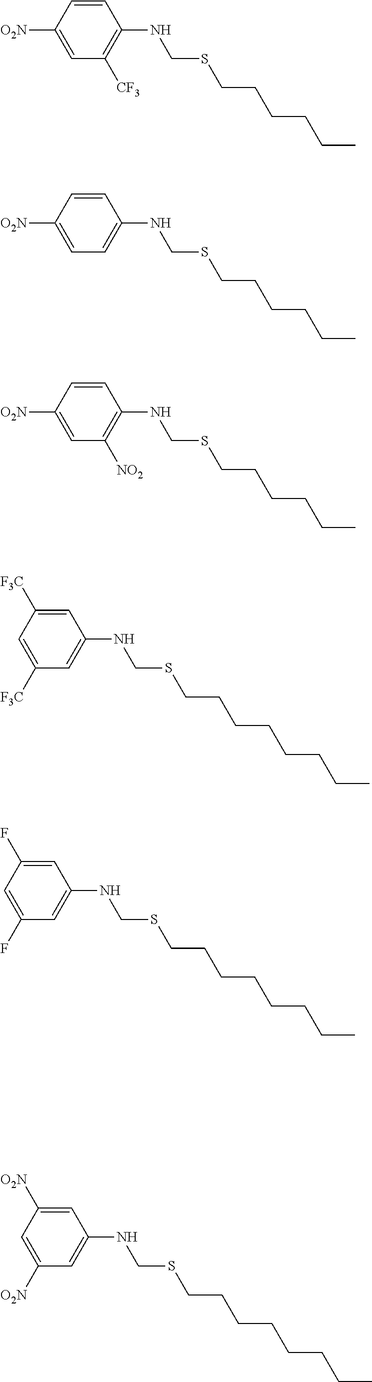 [SMILES: CCCCCCSCNc1ccc([N+](=O)[O-])cc1C(F)(F)F ; CCCCCCSCNc1ccc([N+](=O)[O-])cc1 ; CCCCCCSCNc1ccc([N+](=O)[O-])cc1[N+](=O)[O-] ; CCCCCCCCSCNc1cc(C(F)(F)F)cc(C(F)(F)F)c1 ; CCCCCCCCSCNc1cc(F)cc(F)c1 ; CCCCCCCCSCNc1cc([N+](=O)[O-])cc([N+](=O)[O-])c1]

21. The compound of claim 16, which is selected from the group consisting of:

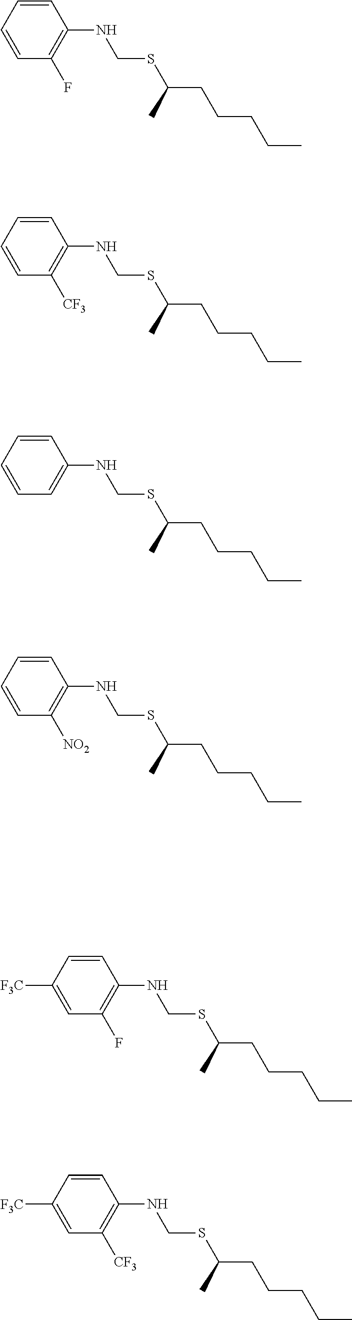 [SMILES: CCCCC[C@@H](C)SCNc1ccccc1F ; CCCCC[C@@H](C)SCNc1ccccc1C(F)(F)F ; CCCCC[C@@H](C)SCNc1ccccc1 ; CCCCC[C@@H](C)SCNc1ccccc1[N+](=O)[O-] ; CCCCC[C@@H](C)SCNc1ccc(C(F)(F)F)cc1F ; CCCCC[C@@H](C)SCNc1ccc(C(F)(F)F)cc1C(F)(F)F]

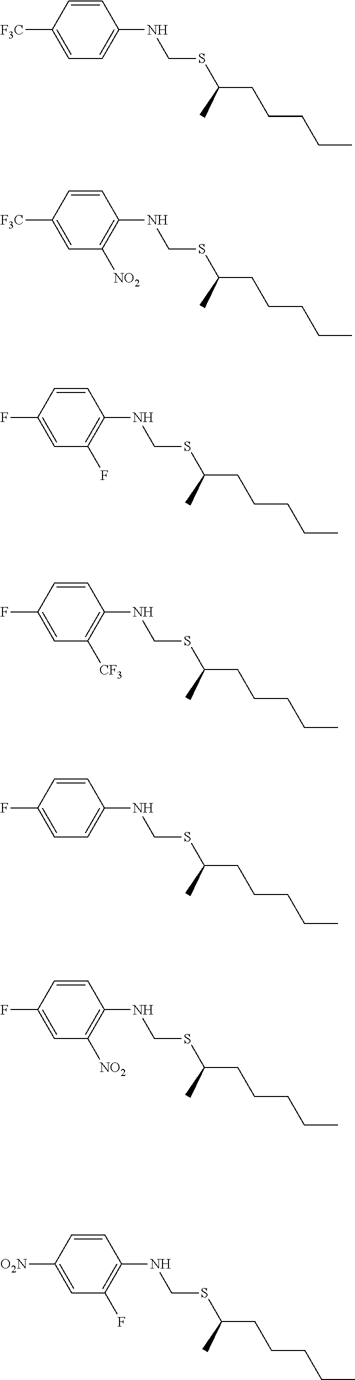

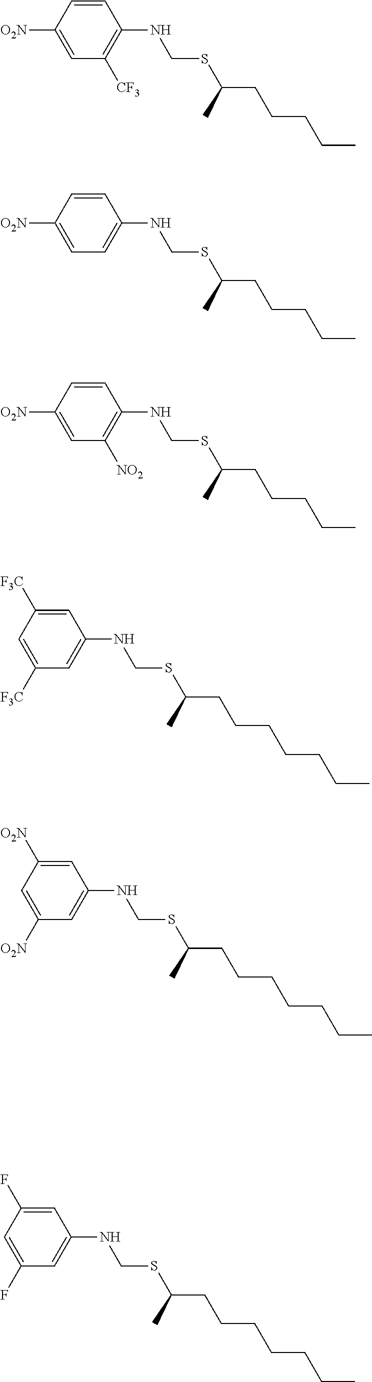 [SMILES: CCCCC[C@@H](C)SCNc1ccc([N+](=O)[O-])cc1C(F)(F)F ; CCCCC[C@@H](C)SCNc1ccc([N+](=O)[O-])cc1 ; CCCCC[C@@H](C)SCNc1ccc([N+](=O)[O-])cc1[N+](=O)[O-] ; CCCCCCC[C@@H](C)SCNc1cc(C(F)(F)F)cc(C(F)(F)F)c1 ; CCCCCCC[C@@H](C)SCNc1cc([N+](=O)[O-])cc([N+](=O)[O-])c1 ; CCCCCCC[C@@H](C)SCNc1cc(F)cc(F)c1]

22. The compound of claim 16, which is selected from the group consisting of:

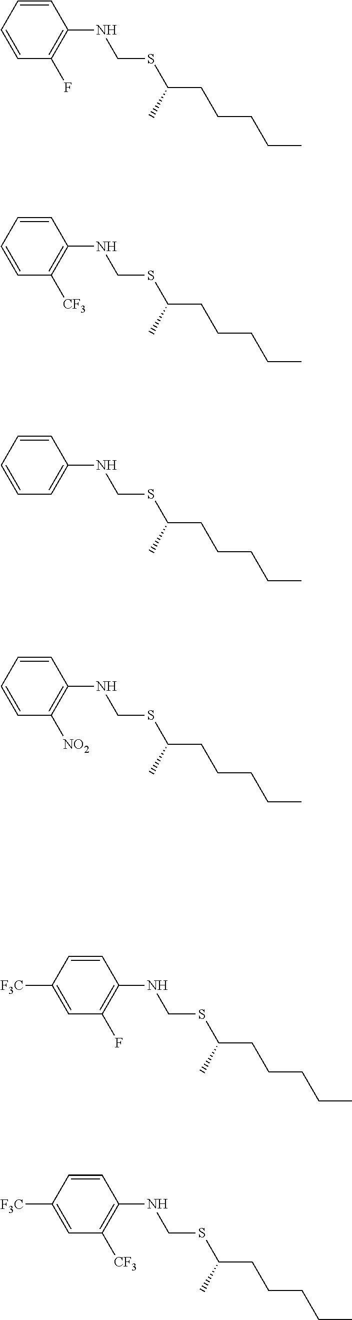 [SMILES: CCCCC[C@H](C)SCNc1ccccc1F ; CCCCC[C@H](C)SCNc1ccccc1C(F)(F)F ; CCCCC[C@H](C)SCNc1ccccc1 ; CCCCC[C@H](C)SCNc1ccccc1[N+](=O)[O-] ; CCCCC[C@H](C)SCNc1ccc(C(F)(F)F)cc1F ; CCCCC[C@H](C)SCNc1ccc(C(F)(F)F)cc1C(F)(F)F]

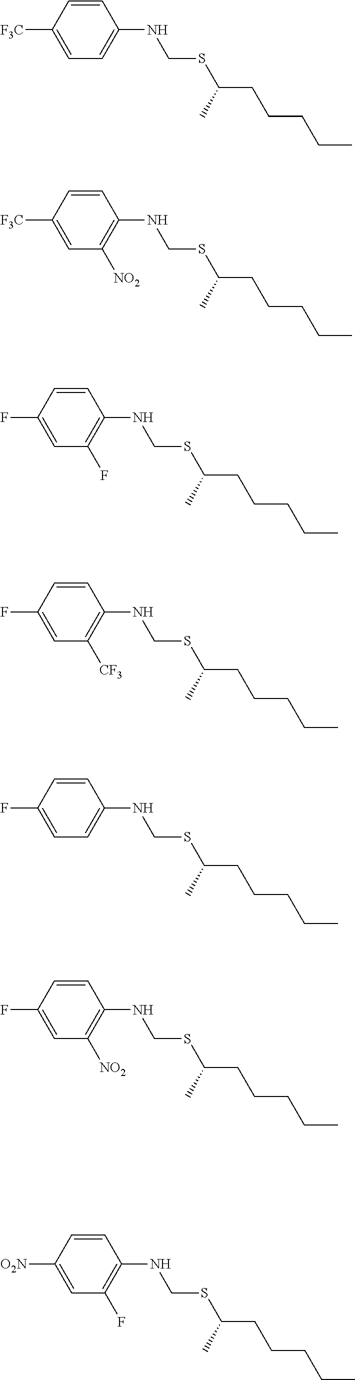

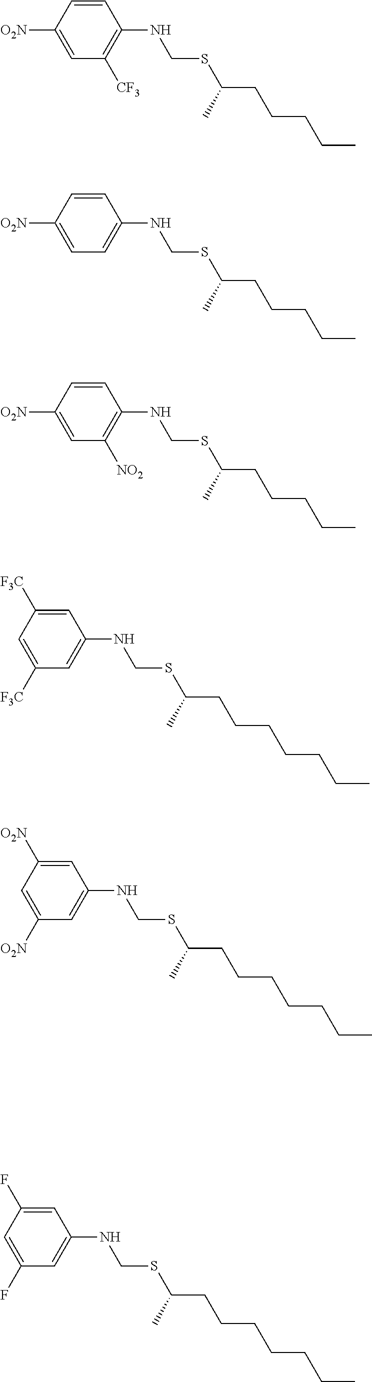 [SMILES: CCCCC[C@H](C)SCNc1ccc([N+](=O)[O-])cc1C(F)(F)F ; CCCCC[C@H](C)SCNc1ccc([N+](=O)[O-])cc1 ; CCCCC[C@H](C)SCNc1ccc([N+](=O)[O-])cc1[N+](=O)[O-] ; CCCCCCC[C@H](C)SCNc1cc(C(F)(F)F)cc(C(F)(F)F)c1 ; CCCCCCC[C@H](C)SCNc1cc([N+](=O)[O-])cc([N+](=O)[O-])c1 ; CCCCCCC[C@H](C)SCNc1cc(F)cc(F)c1]

23. The compound of claim 16, which is selected from the group consisting of:

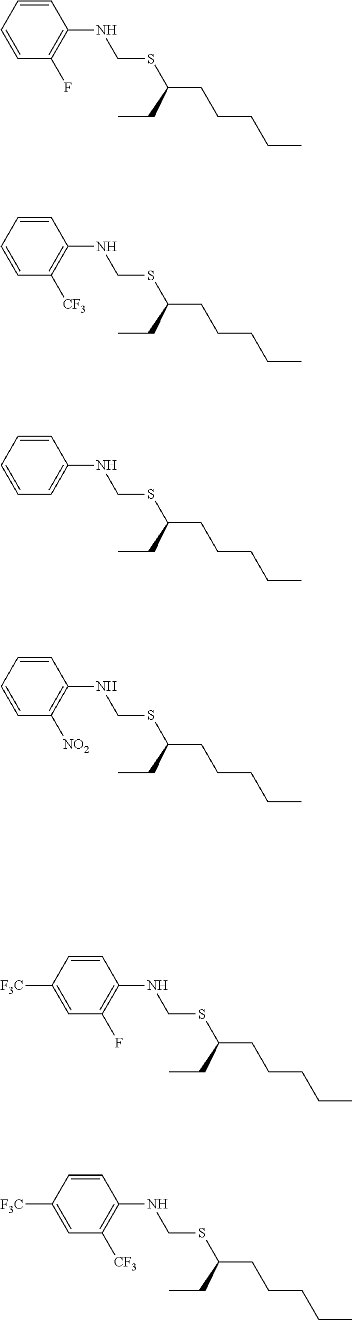 [SMILES: CCCCC[C@@H](CC)SCNc1ccccc1F ; CCCCC[C@@H](CC)SCNc1ccccc1C(F)(F)F ; CCCCC[C@@H](CC)SCNc1ccccc1 ; CCCCC[C@@H](CC)SCNc1ccccc1[N+](=O)[O-] ; CCCCC[C@@H](CC)SCNc1ccc(C(F)(F)F)cc1F ; CCCCC[C@@H](CC)SCNc1ccc(C(F)(F)F)cc1C(F)(F)F]

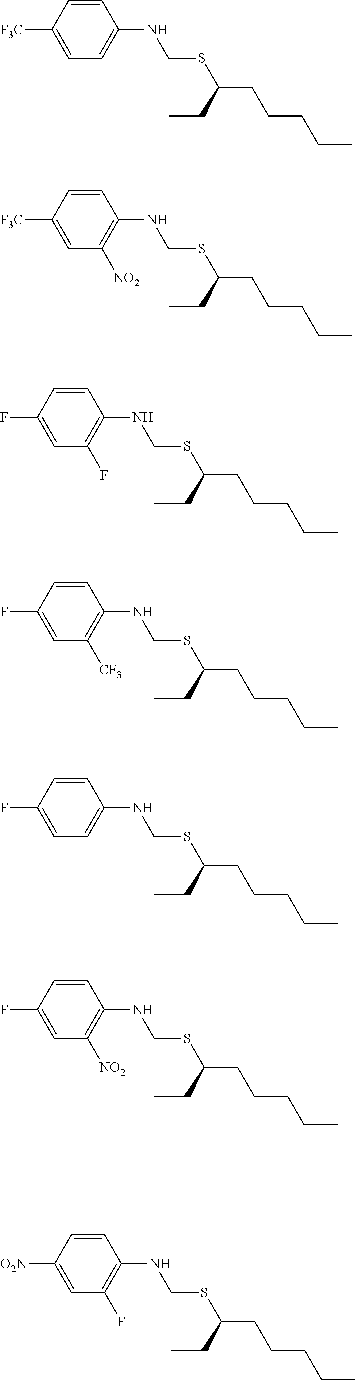

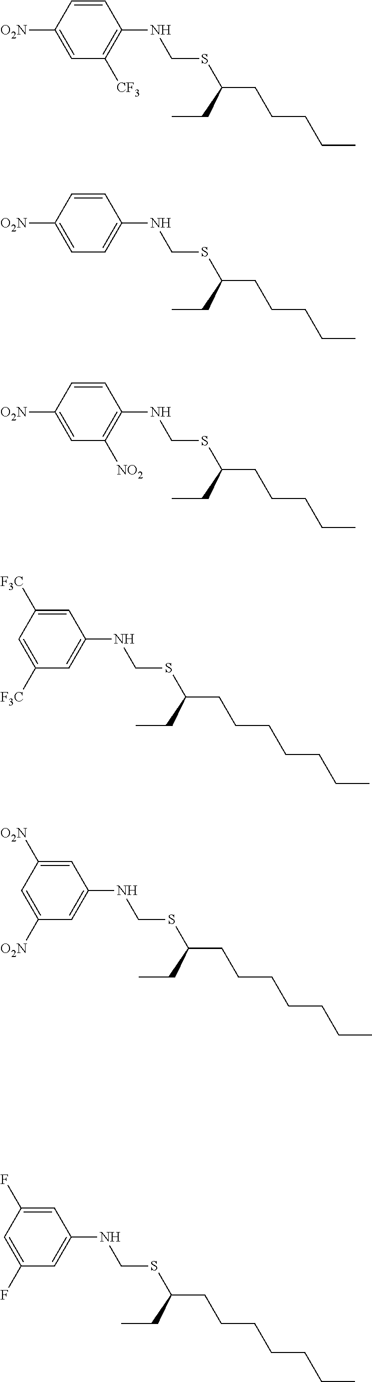 [SMILES: CCCCC[C@@H](CC)SCNc1ccc([N+](=O)[O-])cc1C(F)(F)F ; CCCCC[C@@H](CC)SCNc1ccc([N+](=O)[O-])cc1 ; CCCCC[C@@H](CC)SCNc1ccc([N+](=O)[O-])cc1[N+](=O)[O-] ; CCCCCCC[C@@H](CC)SCNc1cc(C(F)(F)F)cc(C(F)(F)F)c1 ; CCCCCCC[C@@H](CC)SCNc1cc([N+](=O)[O-])cc([N+](=O)[O-])c1 ; CCCCCCC[C@@H](CC)SCNc1cc(F)cc(F)c1]

24. The compound of claim 16, which is selected from the group consisting of:

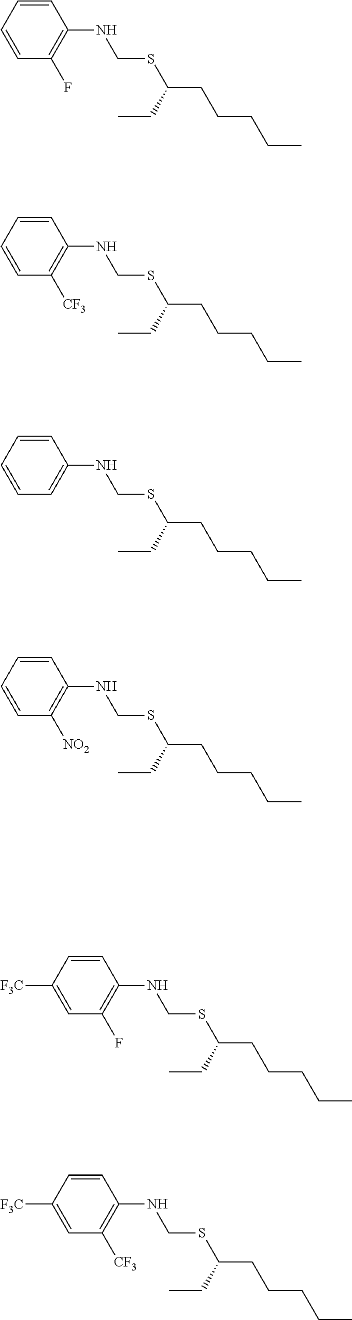 [SMILES: CCCCC[C@H](CC)SCNc1ccccc1F ; CCCCC[C@H](CC)SCNc1ccccc1C(F)(F)F ; CCCCC[C@H](CC)SCNc1ccccc1 ; CCCCC[C@H](CC)SCNc1ccccc1[N+](=O)[O-] ; CCCCC[C@H](CC)SCNc1ccc(C(F)(F)F)cc1F ; CCCCC[C@H](CC)SCNc1ccc(C(F)(F)F)cc1C(F)(F)F]

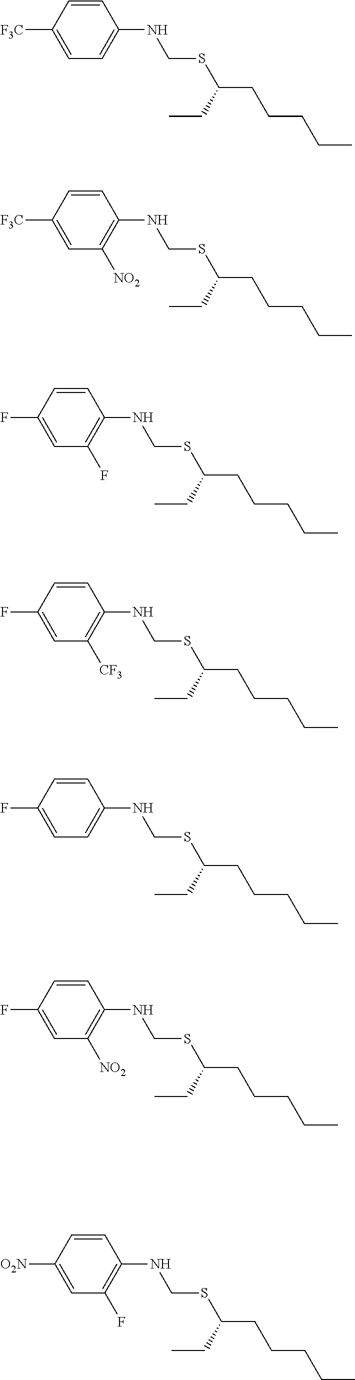

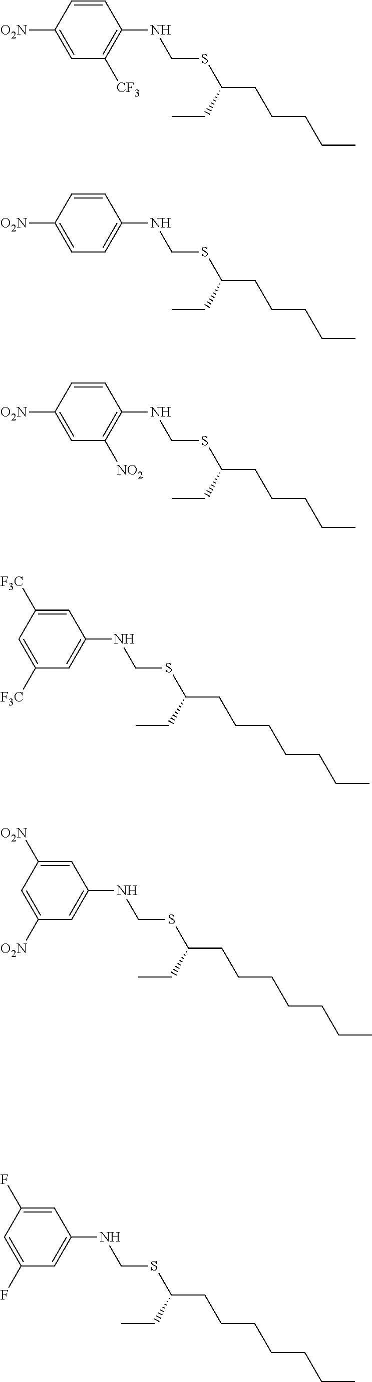 [SMILES: CCCCC[C@H](CC)SCNc1ccc([N+](=O)[O-])cc1C(F)(F)F ; CCCCC[C@H](CC)SCNc1ccc([N+](=O)[O-])cc1 ; CCCCC[C@H](CC)SCNc1ccc([N+](=O)[O-])cc1[N+](=O)[O-] ; CCCCCCC[C@H](CC)SCNc1cc(C(F)(F)F)cc(C(F)(F)F)c1 ; CCCCCCC[C@H](CC)SCNc1cc([N+](=O)[O-])cc([N+](=O)[O-])c1 ; CCCCCCC[C@H](CC)SCNc1cc(F)cc(F)c1]

25. An adduct of the compound of claim 14 of the structure: 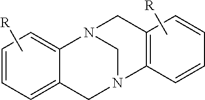
wherein each instance of R is independently selected from the group consisting of —H, —F, —CF₃, —NO₂ and —N(Me)₂.

26. A catalyst system comprising 1,8-Diazabicycloundec-7-ene and the compound of claim 25.

27. -28. (canceled)

29. A reaction mixture comprising the compound of claim 25 and one or more monomers selected from the group consisting of lactide, carbonate, and ester.

30. The compound of claim 25, which is selected from the group consisting of:

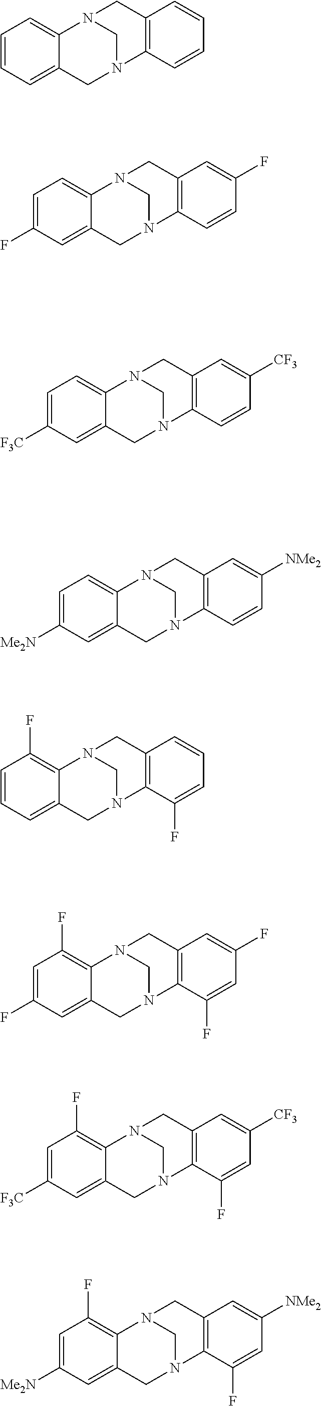

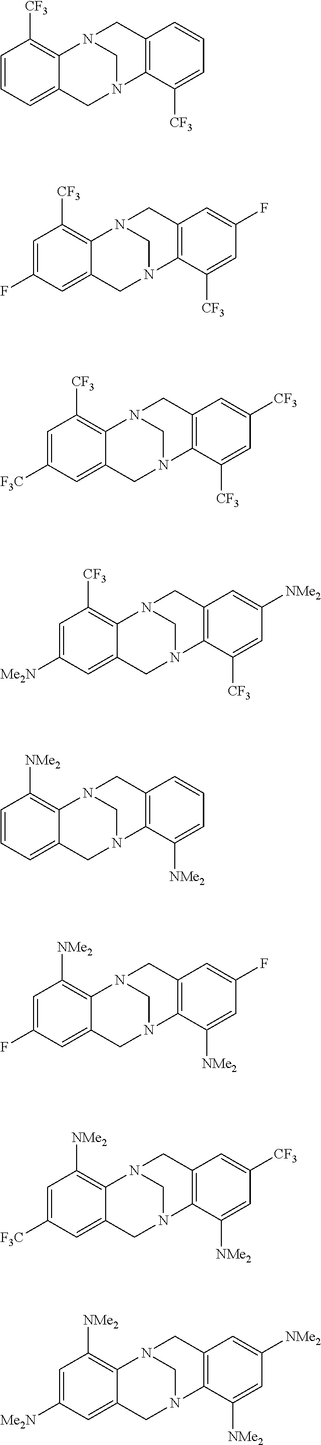

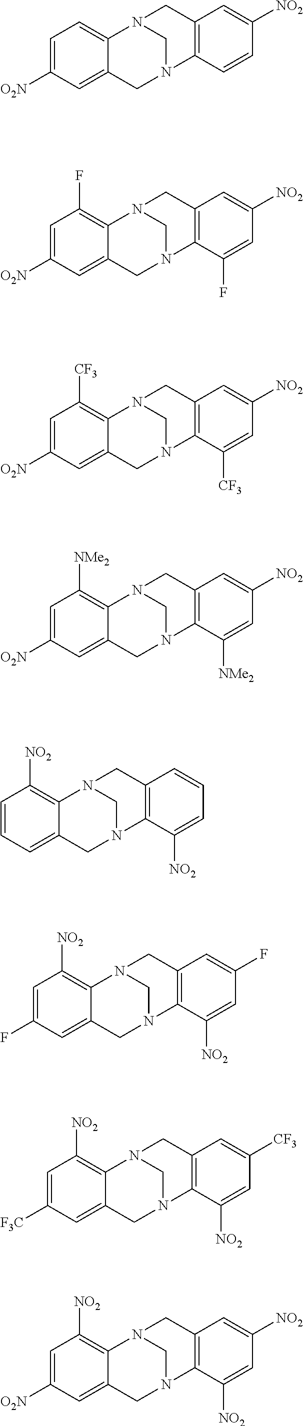

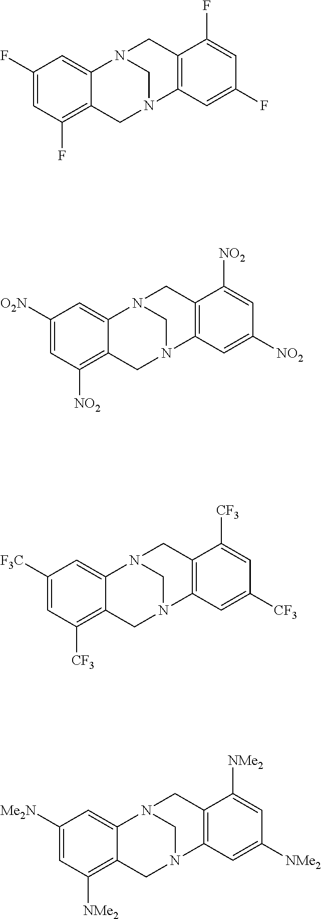 [SMILES: Fc1cc(F)c2c(c1)N1Cc3c(F)cc(F)cc3N(C2)C1 ; O=[N+]([O-])c1cc2c(c([N+](=O)[O-])c1)CN1CN2Cc2c1cc([N+](=O)[O-])cc2[N+](=O)[O-] ; FC(F)(F)c1cc2c(c(C(F)(F)F)c1)CN1CN2Cc2c1cc(C(F)(F)F)cc2C(F)(F)F ; CN(C)c1cc(N(C)C)c2c(c1)N1Cc3c(N(C)C)cc(N(C)C)cc3N(C2)C1]

31. A method of polymerizing a monomer comprising:
mixing a monomer and a catalyst to form a polymer, wherein the monomer is selected from the group consisting of lactide, carbonate, ester, and mixtures thereof, wherein the polymer has a polydispersity index between about 0.8 to about 1.5, wherein the polymer has an Mn or Mw between about 5,000 g/mol to about 25,000 g/mol, and wherein the catalyst is selected from the group consisting of: 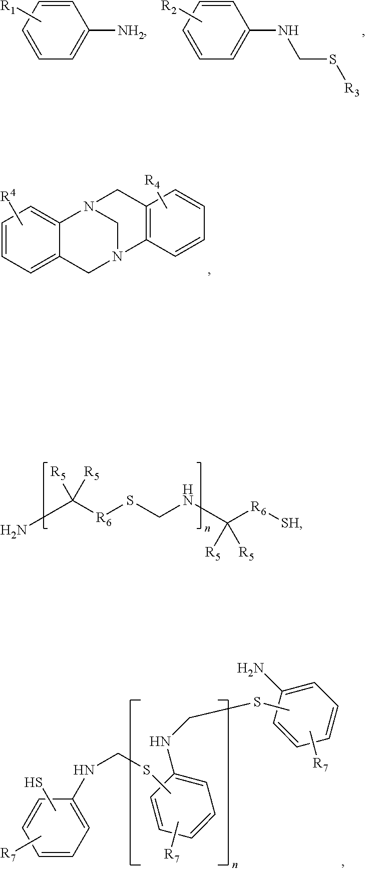
and mixtures thereof,
wherein each instance of R₁ is independently selected from the group consisting of —H, —F, —CF₃, —NO₂, —Cl, —Br, —I, nitrile, and —N(alkyl)₂,
wherein each instance of R₂ is independently selected from the group consisting of H, —F, —CF₃, —NO₂, —Cl, —Br, —I, and nitrile,
wherein R₃ is selected from the group consisting of linear or branched alkyl,
wherein each instance of R₄ is independently selected from the group consisting of —H, —F, —CF₃, —N(Me)₂, —NO₂,
wherein each instance of R₅ is independently selected from the group consisting of —H, —F, —CF₃, —NO₂, —Cl, —Br, —I, and nitrile,
wherein R₆ is selected from the group consisting of cycloalkyl, alkyl, alkylene glycol, acrylate, and siloxane, and
wherein each instance of R₇ is independently selected from the group consisting of —H, —F, —CF₃, —NO₂, —Cl, —Br, —I, nitrile.

32. The method of claim 31, further comprising mixing a co-catalyst with the catalyst to form a catalyst system.
